SEQUENCE LISTING (9 sequences)

SEQ ID NO: 1 - DNA, 2505 bp, Homo sapiens
  human UC28 splice variant cDNA
gaccttaaatatatcgaggtggctaattgatgtataataatttacaaaattattcttctattgctacagagctacaattcaatttacagtaggccaccatgagggccttcttaaggaaccagaaatatgaggatatgcacaatattattcacattttacagatcagaaaattgaggcacagattaagtaacttcccaaggctaccaggcattctagctccagaaactgtgctcttaccattctgctacaaggtatttcgaaaaaaagaaaaagtaaaaagaagtcaaaaggcaacagagttcattgattattccatagaacagtcacaccatgcaattctcacacccttgcagacacacttgaccatgaaaggttcctcaatgaaatgttcctcattatcttcagaagccatattattcacattgactttgcagttaactcagaccctaggtctggaatgctgtcttctctacttatccaaaactatacatccacagatcatataaactctcagccctgctgcaaagcctttccagaaaaataaaaatggttgaaaaggcaattctgctaccaatgactgtttaagcccagccaagtaactgaaccattccaacttcaatttacttatgaaaagaatttgatgatgtaggaggttatttcaattctaaaatacaaacccatgttgatctttctcaatcttgaactcatagattattatctattatctcaatttagtttgttatttatcctagtgggccattaaaaactaccacatgtgtttctgtctctccattagtcaataactaaactaacgagcaattagtaagccatgtgccagatgctccgctaggcaccagagggataaaaacaatacttatagtataccactaattttcgcttagtaactagtgaaatgttcaagtcatgcctgagtcaagagttgaggagacattacaatgtgtaatggaaaccaaggaaagtgaaactttggataagtggggactagtgtatttatatatttaattgatttctgactctatcattggcctccaaacacagattgtgtttttctttggttttgttttcttcactatgggatcttctgtgcccagcacagtgcctgacacatagaaaacaatcaatatttgctgaataaatgattaaaaaatcagagaactttcccattctgtttggatctatagaacatccagagtaagtgatgagggcctctgcatttatatgcgcttaaattaagattatgtgagaaaagtttaaagacacttagtagagtgattttgaaatatagtaaacacttggaaatggtggtgctttaaaaagatattaatagataatatgaaaatctccatctcaaaaataatgcataaactatttaaaggaaaatcacatctccaggctttcaatgtttgttcattactttttcatatatttttaccatctgctgaaggcagtcatatcaaagggtaaagaaagatgggaggaaaactcagtaagaattatattagtctgtttgcaaagtagaaaaagattctcatcactcaaccttatgagcaggaagagggaaggctgtttgagaaccatttacttagcagaaccacatattttagacacttccctgcattaactgcacaaacaatatgtttgcaaacttgttrgatcaacctccaacaacgacacattcaggagttaaatatttttcatcaaacattggatttttccttaacgctagagattgctacaaatcttctgaagggtctcaatggcttcaggctaagaagagatttctccctgttataagcagcaagacaaattagccatttcactctcaaacttcactaatgatcacattctttccaaaaggaactctagaagaccaaatgccccgagttaagaacatcaaaactaaccatctgaagaaacttcccaagtgtaagactctgccattaaaacattaccgagaggggactcaaacagtctttcttcctttgtcgtgtttcttgctcccagaccaaggcactgacgacagtactgatacataatttaaaagcacactcccttccactttggtaataccagaactctaattggaccaccctgaagcttaggactaccagccatacaaatagtaaactctgtccacgattcactcatctgtgtattttctatagatgtttactaggcgtttgttatataaaaataccccggccaggcacggtggctcacgcctgtaatcccagcactttgggaggtgggtggatcacctgaggtcgggagttcgagaccagcctgaccagcatggtggaacccccatctctactaaaaacacaaaaaattagccgggcgtggtggcacatgcctgtaatcccagctactcaggaggctgaggcggagaattgcttgaacccggaaggtggaggttgttgcggtgagctgagattgcactattgcactccagcctgggcaacaggagtaaaactcccccccacc

SEQ ID NO: 2 - protein, 135 aa, Homo sapiens
MRAFLRNQKYEDMHNIIHILQIRKLRHRLSNFPRLPGILAPETVLLPFCYKVFRKKEKVKRSQKATEFIDYSIEQSHHAILTPLQTHLTMKGSSMKCSSLSSEAILFTLTLQLTQTLGLECCLLYLSKTIHPQII

SEQ ID NO: 3 - protein, 21 aa, Artificial Sequence
  synthetic UC28 peptide UC28A 1
RKKEKVKRSQKATEFIDYSTE

SEQ ID NO: 4 - protein, 17 aa, Artificial Sequence
  synthetic UC28 peptide UC28C 1
QIRKLRHRLSNFPRLPG

SEQ ID NO: 5 - DNA, 175 bp, Artificial Sequence
  synthetic truncated neu,  truncated NEU
acccactcgtgagtccaacggtcttttctgcagaaaggaggactttcctttcaggggtctttctggggctcttactataaaaggggaccaactctccctttgtcatatcttgtttctgatgacaaaaaataacacattgttaaaattgtaaaattaaaacatgaaatataaatta

SEQ ID NO: 6 - DNA, 166 bp, Homo sapiens
  human UC 38
gtttcgctccacattcatcctttcttactgggcactgatgttgagagcatcaggcagggtataatgttatgttgcagtaacaaacaccctcaatatctcagtggcttaaaatgacaacgatcttttttttgtttgtttgtttatgctctatatcacccagggatca

SEQ ID NO: 7 - DNA, 183 bp, Homo sapiens
  human UC 41
caaccttagcccctctcctcttcttcacgatgccattctgccatttctgttttgtggtagacaggttggcccaggcactctaaggcccaggctggcacaggttggcccaggcacttcaagcctaagtccatttacagtttctattccatctcttcctaaagaagaggagaggggctaaggttg

SEQ ID NO: 8 - DNA, 673 bp, Homo sapiens
  human UC 31
caggacacagagtaagatacccactgacttcttgtggtctacttcctgggtgttgtttcaatgggctttgttataacaggactagtcttctgtaaatacaacttggtaaataggatgaaacataactttgcgacaattcagtagaaataggcatacaaacctgggcctgatgacactcacctccccttggctataaacattaccctacctgttaagtcagtaatcctttgggagagcgcttactgagtatctatgatatgcaaagaccaaagaccgagggggatccctggtgtagagcaagcacacacctggttattagctacctgccaccctgctgggcatgcaacatacattgtctcaaattctaaccaccctgcaaggcaagcttccttgttcttttaaagaagaaaagtagaccagcaagattgatttgctcaagattacacagcctggaatcttgtcatgggcatgtctgactctgatagcaataccctcaaagaaactgtcagagaagactcaataagaagaaagttgagatacagaaaccaacaggagaaggtaattcagaaattcaaacagagtgggtgtgatgggaagaattcattaataagaaggtacctctgtagaaaaatcttaccagacagtctggaagtgaaggaaacagccaatagtc

SEQ ID NO: 9 - protein, 4 aa, Artificial Sequence
  synthetic peptide spacer, spacer arm
LALA

(19) United States
(12) Patent Application Publication
(10) Pub. No.: US 2012/0213789 A1
(43) Pub. Date: Aug. 23, 2012

(54) New Molecular Target for Treatment of Cancer

(76) Inventors: Gang An, Durham, NC (US); S. Mark O'Hara, Ambler, PA (US); David Ralph, Edmond, OK (US); Robert W. Veltri, Oklahoma City, OK (US)

(21) Appl. No.: 13/328,565

(22) Filed: Dec. 16, 2011

Related U.S. Application Data
(63) Continuation of application No. 09/966,762, filed on Sep. 28, 2001, now abandoned.

(51) Int. Cl. A61K 39/395; A61P 35/04; A61P 35/00; G01N 33/574; C12Q 1/68

(52) U.S. Cl. 424/139.1; 436/501; 435/6.11

(57) ABSTRACT

The present invention concerns preventative, therapeutic, and diagnostic methods and compositions involving UC markers, such as UC 28, for cancer. It includes methods and compositions for targeting cancer cells using a differentiation agent in combination with a therapeutic agent targeted to cells that differentially express a UC marker after exposure to the differentiation agent. The invention also includes methods of inducing immune responses against UC markers, as well as antibodies that recognize UC markers, which may be employed for therapeutic and diagnostic methods.

### CROSS-REFERENCE TO RELATED APPLICATIONS

This application is a continuation of co-pending application U.S. patent application Ser. No. 09/966,762, filed Sep. 28, 2001, the disclosure of which is incorporated herein by reference in its entireties.

### BACKGROUND OF THE INVENTION

### A. Field of the Invention

The present invention relates generally to the field of oncology. More particularly, it concerns preventative and therapeutic methods and compositions involving UC markers, including UC28, and modulators thereof. Furthermore, the invention concerns agents that target cancer cells.

### B. Reference to a “Sequence Listing,” a Table, or a Computer Program Listing Appendix Submitted as an ASCII Text File

The Sequence Listing written in file UROC033USC1.TXT, created on Dec. 16, 2011, 12,288 bytes, machine format IMB-PC, MS-Windows operating system, is hereby incorporated by reference in its entirety for all purposes.

### C. Description of the Related Art

Cancer is the second leading cause of death in the United States producing 38, 500 deaths in year 2000. Half of all men and one-third of all women in the US will develop cancer during their lifetimes. Today, millions of people are living with cancer or have had cancer.

The four major types of treatment for cancer are surgery, radiation, chemotherapy, and biologic therapies. All these therapies have side effects and very importantly often lead to killing of normal cells apart from the cancerous cells. This is because these therapies are non specific and cannot distinguish between a healthy and a cancerous cell.

Differentiation therapy is a new approach to the treatment of advanced or aggressive malignancies and may show significant efficacy in the treatment of cancer. To understand the principles and rationale of differentiation therapy, one needs to understand the origins of the cancer cell, which evolves through a process of carcinogenesis. Cancer cells are essentially normal cells that, through a series of environmental and/or genetic alterations, have regressed to a more immature or less differentiated state. As a direct result of this transformation, these cells have lost the ability to control their own growth, a control mechanism that normal mature cells possess. Consequently, the affected cell multiplies at an abnormally fast rate, invades into blood vessels and lymphatic channels, and spreads throughout the body unchecked. The application of differentiation therapy seeks to reverse this loss of the differentiated state and force the cancer cell to resume a more mature phenotype. The application of differentiation therapy halts the progression of the cancer, allowing the transformed cells to regain the appearance and cell functions of a mature cell, much like a cell from the organ where the cancer cell originated.

While this would not eradicate the cancer, it would stop the growth of the tumor and halt metastatic progression, allowing the application of more conventional therapies to eradicate any cancerous growths. If differentiation therapy were applied early enough in the evolution of a cancer, one could circumvent the growth of a tumor without the need for additional therapy. There may be a role for differentiation therapy as a chemopreventive strategy, whereby patients at risk for the development of malignancy could take differentiation agents as prophylaxis against the development of cancer. By and large, the differentiation agents studied to date have demonstrated significantly less toxicity as compared to standard cancer treatments. Because of this low toxicity profile, differentiation therapy could be employed effectively as chemoprevention for cancer in selected circumstances.

Among the various anticancer drugs used, differentiation-inducing agents have been used to induce differentiation of carcinoma cells for controlling their infinite proliferation, rather than directly killing the cells. These agents may, be inferior to the anticancer drugs that directly kill carcinoma cells but may be expected to have reduced toxicity and differential selectivity. In fact, it is well known that retinoic acid, a differentiation-inducing agent, may be used for treatment of acute promyelogenous leukemia to exhibit a higher toxic effect (Huang et al., 1988, Castaign et al., 1990; Warren et al., 1991). In addition, vitamin D derivatives exhibit differentiation-inducing effect, and thus their application for anticancer drugs have been investigated (Olsson et al., 1983).

As the results of these investigations, there have been reported applications for anticancer drugs, of a variety of differentiation-inducing agents such as vitamin D derivatives (JP-A 6-179622), isoprene derivatives (JP-A6-192073), tocopherol (JP-A6-256181), quinone derivatives (JP-A 6-305955), noncyclic polyisoprenoids (JP-A 6-316520), benzoic acid derivatives (JP-A 7-206765) and glycolipids (JP-A 7-258100).

Further, retinoids and short-chain fatty acids have shown biological activity as single agents in several preclinical studies of different tumors (Lippman and Davies, 1997; Dahiya et al., 1994; Samid et al., 1992; Samid et al., 1997). Aliphatic and aromatic fatty acids such as sodium butyrate (SPB), and its metabolite phenylacetate have been reported to induce tumor cell cytostasis, differentiation, and apoptosis in various hematological and solid tumors, including prostate cancer (Carducci et al., 1996; Melchior et al., 1999). Differentiation-inducing agents, such as retinoids and short-chain fatty acids, have an inhibitory effect on tumor cell proliferation and tumor growth in preclinical studies. Clinical trials involving these compounds as single agents have been suboptimal in terms of clinical benefit.

Thus, there continues to be a need for a cancer treatment that is non-toxic, yet has the highly specific qualities of a differentiation agent and the powerful cell-killing characteristics of an anticancer agents or anticancer therapy in order to achieve a therapy that selectively kills cancerous cells.

### SUMMARY OF THE INVENTION

The present invention concerns UC markers, identified by SEQ ID NO in U.S. Pat. No. 6,218,529, and their use for diagnostic, preventative, and therapy methods concerning cancer. It takes advantage of the observation that RNA expression of UC markers is increased in cancer cells compared to normal or noncancerous cells and that differentiating agents effect an increase in UC markers, such as UC28, in cancer cells compared to normal cells. It is specifically contemplated that the methods, compounds, and compositions discussed below may be implemented with one another interchangeably.

The present invention, in some embodiments, concerns methods for inhibiting a cancer cell that expresses a UC marker by administering to the cell an effective amount of a composition comprising a UC marker inhibitor. The term “administering” means to give or apply, and it includes providing or contacting a cell or patient with a particular compound, agent, or composition. In additional embodiments, the method also includes administering to the cell a differentiation agent that increases the level of a UC marker against which the inhibitor is directed or targeted.

A UC marker inhibitor is a substance that inhibits or reduces the activity or function of a UC marker or a substance (also referred to as “UC marker targeted inhibitor”) that uses the UC marker as a target to inhibit the cell that expresses the UC marker or a cell adjacent to that cell (bystander effect). A cell that is inhibited may, for example, have its growth rate reduced, it may be induced to undergo apoptosis, it may not divide anymore, it may die, or it may be more amenable to inhibition by other therapies such as chemotherapy or radiotherapy. An inhibitor of function or activity includes, but is not limited to, compounds that bind the UC marker, reduce the expression of UC marker RNA transcripts, reduce the stability of the UC marker or UC marker transcript, decrease the half-life of the UC marker or UC marker transcript, alter the localization of the UC marker or the UC marker transcript, decrease the availability of the UC marker or the transcript, alter the processing of the UC marker or transcript, or modify the UC marker or transcript so that it can no longer interact with a substance that acts immediately upstream or downstream of it in any series of interactions with which it may be involved.

In other embodiments, methods of the invention concern treating a patient with cancer by administering to the patient a composition comprising a UC marker inhibitor. In additional embodiments, methods further include administering to the cell a differentiation agent that increases the level of the UC marker against which the inhibitor is directed or targeted.

UC markers that may be employed in any methods or compositions of the invention include all or part of any nucleic acid or amino acid disclosed with a SEQ ID NO in U.S. Pat. No. 6,218,529, which is specifically incorporated by reference. It is specifically contemplated that UC28, UC31, UC38, UC41, and the truncated neu may be used as the UC marker in the methods described. In some embodiments, UC28 is the UC marker being targeted or inhibited.

UC marker inhibitors include a variety of compounds. In some embodiments, the inhibitor specifically binds a UC marker. It is contemplated that the inhibitor may be a small molecule, a nucleic acid molecule or a proteinaceous composition, such as a polypeptide or peptide. In some embodiments, the inhibitor is a polypeptide. In other embodiments, the inhibitor is an antibody, either a polyclonal or a monoclonal antibody. A polyclonal antibody UC28A 1 or UC28C1 are exemplary polyclonal antibodies. UC28A 3-1 G2, UC28A 1-4 A3, UC28A 3-3 G10, UC28A 1-4 C9, UC28A 4-1 H5, UC28C 2-2 D2, UC28C 1-1 A1, UC28C 1-1 A2, UC 28C 3-1F3, or UC 28C 2-3 G2 are exemplary monoclonal antibodies.

In still further embodiments, the inhibitor is a fusion or chimeric protein. A fusion or chimeric protein, in some embodiments, contains a targeting moiety and an effector moiety whereby the targeting moiety allows the effector moiety to inhibit a particular cell that is recognized by the targeting moiety. Such a protein may include, for example, all or part of a toxin or all or part of an antibody. In some aspects of the invention, the toxin is a ribosome inhibitory protein or an apoptosis inducing agent. Ribosome inhibitory proteins include abrin, diptheria toxin, gelonin, mitogillin, pseudomonas exotoxin, ricin A chain, saporin, and shiga toxin, and embodiments concern all or part of at least one of these toxins, or a combination thereof. An apoptosis inducing agent is a compound that induces apoptosis of a cell when introduced into or contacted with a cell. Such agents include, but are not limited to, BAD, Bax, TNFα, TNFβ, Fas-L, p53, Myc, or onconase. It is contemplated that proteinaceous compositions of the invention may be produced using or administered as a nucleic acid encoding the composition.

In further embodiments the UC marker inhibitor, such as a UC28 inhibitor, is a nucleic acid molecule with a sequence identical or complementary to all or part of a UC marker-encoding nucleic acid. The inhibitor may be identical or complementary to all or part of SEQ ID NO:1, SEQ ID NO:5, SEQ ID NO:6, SEQ ID NO:7, or SEQ ID NO:8. In some aspects of the invention, the nucleic acid molecule is a ribozyme, while in other aspects, it is an antisense molecule.

Nucleic acids of the invention may be comprised in an expression construct comprising a nucleic acid sequence encoding the nucleic acid molecule. In some embodiments, a cell or a patient is administered an expression construct encoding an UC marker inhibitor. The expression construct will be engineered to achieve or provide expression of the UC marker inhibitor in the cancer cells or cells helping to sustain the cancer cells, such as vascular cells helping to feed a tumor. It is contemplated that the expression construct may be a viral vector, such as an adenovirus vector, an adeno-associated virus vector, a herpesvirus vector, a lentivirus vector, a retrovirus vector, a vaccinia virus vector.

Methods of the invention also involve compositions that include, in addition to a UC marker inhibitor, one or more lipid molecules. Different types of lipid molecules may be employed, depending on whether the composition comprises nucleic acids or proteinaceous compositions.

The cancer cell being inhibited or treated includes, but is not limited to, a bladder cell, a breast cell, a lung cell, a colon cell, a prostate cell, a liver cell, a pancreatic cell, a stomach cell, a testicular cell, a brain cell, an ovarian cell, a lymphatic cell, a skin cell, a bone cell, or a soft tissue cell. In some embodiments, the cancer cell being inhibited or treated is in a patient. UC marker inhibitors, differentiation agents, vaccine compositions, or other compounds of the invention may be administered to a cell or patient directly, regionally, parentally, orally, intravenously, intraperitoneally, intratracheally, intramuscularly, intratumorally, subcutaneously, endoscopically, intralesionally, percutaneously, or by direct injection. Compounds or compositions may be administered multiple times, such as 2, 3, 4, 5, 6, 7, 8, 9, 10 or more times. They may be administered every 1, 2, 3, 4, 5, 6, 7, 8, 9, 10, 11, 12, 13, 14, 15, 16, 17, 18, 19, 20, 21, 22, 23, 24 or more hours, every 1, 2, 3, 4, 5, 6, 7, or more days, or every 1, 2, 3, 4, 5, 6, 7, 8, 9, 10, 11, 12 or more weeks. Subsequent administrations may also occur after such time periods have gone by. Also, such periods of time may occur between administration of different agents. For example, there may be 4 hours lapse between administration of a differentiating agent and an UC marker inhibitor. Furthermore, in some embodiments, different agents are administered at the same time. A differentiating agent may be administered at the same time as a UC marker inhibitor. Alternatively, the differentiating agent may be administered before or after the UC marker inhibitor.

In some embodiments of the invention, a differentiation agent may be used. The differentiating agent may be, for example, sodium phenylbutyrate (SPB), sodium phenylacetate, retinoid, a short chain fatty acid, DMSO, n-methylformamide, vitamin D3, a vitamin D3 analog, vitamin E, an estrogen, a glucocorticoid, a protein kinase C(PKC) activator, a PKC inhibitor, thiazolidinedione-including troglitazones-oxacalcitriol, onconase, and analogs thereof. It is contemplated that the differentiation agent may be administered to cells or to a patient at a concentration of between 0.1 mM and 500 mM, between 0.2 mM and 100 triM, between 0.5 mM and 25 mM, or between 1 mM and 10 mM. It is contemplated that the concentration of the differentiation agent may be at, be at least, or be greater than 1, 2, 3, 4, 5, 6, 7, 8, 9 10, 11, 12, 13, 14, 15, 16, 17, 18, 19, 20, 21, 22, 23, 24, or 25 mM.

Treatment methods of the invention include, in some embodiments, administering to the patient a second anti-cancer therapy. Exemplary anti-cancer therapies are chemotherapy, radiotherapy, hormonal therapy, gene therapy, or immunotherapy. It is specifically contemplated that chemotherapy and/or radiotherapy may be administered to a patient.

In addition to methods of treating, screening methods also are provided by the present invention. Methods of screening for a modulator of a UC marker include at least the following steps: a) obtaining a cell that expresses the UC marker; b) contacting the cell with a candidate substance; and c) determining the ability of the candidate substance to modulate the UC marker, wherein a modulation in the activity or amount of the UC marker in the cell identifies the candidate substance as a UC marker modulator. Screening for modulators of UC28 is specifically contemplated. The modulator may be an inhibitor of the UC marker or an enhancer of the UC marker. In some embodiments, the method further includes administering to the cell a differentiation agent that increases the amount of the UC marker in the absence of the candidate substance. It is contemplated that the differentiation agent may be administered before, after, or at the same time the candidate substance is provided to the cell or cells. In additional embodiments, the step of comparing the amount or activity of UC marker in the cell contacted with the candidate substance with the amount or activity of UC marker in a cell not contacted with the candidate substance.

In some embodiments, the screening method includes evaluating the cell contacted with the candidate substance for apoptosis. Apoptosis can be evaluated by measuring the expression of compounds involved in apoptosis, for example, Annexin V, Fas, or Bc1-2. Alternatively, it can be determined by DNA fragmentation, or any other sign of apoptosis. In still further embodiments, the ability of the candidate substance to modulate a UC marker is determined by measuring the amount of that UC marker or transcripts encoding that UC marker in the cell. This can be done by using an antibody that specifically recognizes the UC marker or a nucleic acid that is identical or complementary to the UC marker transcript or a UC marker cDNA. In cases in which an antibody is used, the antibody may be a polyclonal antibody such as UC28A 1 or UC28C1. The antibody may also be a monoclonal antibody, such as UC28A 3-1 G2, UC28A 1-4 A3, UC28A 3-3 G10, UC28A 1-4 C9, UC28A 4-1 H5, UC28C 2-2 D2, UC28C 1-1 A1, UC28C 1-1 A2, UC 28C 3-1F3, or UC 28C 2-3 G2.

The present invention also concerns methods for preventing or treating cancer in a patient in which a UC marker is employed as a vaccine. These methods involve administering to the patient a composition comprising a peptide comprising at least 4 contiguous amino acids from a UC marker, so the patient will have an immune response against the peptide. It is contemplated that the UC marker may be, for example, UC28, UC31, UC38, or UC41. It is further contemplated that the peptide comprises contiguous amino acids from SEQ ID NO:2, SEQ ID NO:3, or SEQ ID NO:4. The peptide may be 4, 5, 6, 7, 8, 9, 10, 11, 12, 13, 14, 15, 16, 17, 18, 19, 20, 21, 22, 23, 24, 25 or more contiguous amino acids of SEQ ID NO:2, SEQ ID NO:3, or SEQ ID NO:4.

In further embodiments of the invention, the vaccine composition comprises more than one peptide sequence of at least 4 contiguous amino acids from SEQ ID NO:2 or SEQ ID NO:4. The composition, in some embodiments, also includes one or more different lipids, and it may also have an adjuvant. The peptide of the vaccine composition may also be comprised in a polypeptide conjugate multimer.

Other vaccine compositions of the invention include activated, isolated antigen presenting cells (APC), wherein the cells are stimulated by exposure in vitro to a peptide comprising at least 4 contiguous amino acids of a UC marker wherein the cells are effective to activate a T-cell response against the UC marker in the patient. The peptide may be contiguous amino acids of SEQ ID NO:2, SEQ ID NO:3, or SEQ ID NO:4. In some embodiments, the antigen presenting cells are dendritic cells. It is contemplated that any embodiment with respect to one vaccine composition may be implemented with respect to other vaccine compositions described herein.

Other methods of the invention concern methods for diagnosing cancer in a patient. Such methods involve assaying a sample from a patient for UC28 using an antibody directed against UC28, wherein the detection of an elevated level of UC28 protein compared to normal cells is indicative of cancerous cells. The antibodies may be, for example, UC28A 3-1 G2, UC28A 1-4 A3, UC28A 3-3 G10, UC28A 1-4 C9, UC28A 4-1 H5, UC28C 2-2 D2, UC28C 1-1 A1, UC28C 1-1 A2, UC 28C 3-1F3 or UC 28C 2-3 G2, as well as UC28A 1 or UC28C 1. The antibodies may also specifically recognize or bind SEQ ID NO:3 or SEQ ID NO:4. In some embodiments, the sample is obtained from any tissue suspected of being cancerous, for example, prostate, bladder, or breast tissue. In still further embodiments, the antibody is attached to a detection reagent, which allows the antibody to be detected and/or quantified. The detection reagent can be, for example, colorimetric, radioactive, or enzymatic. With an antibody, the sample may be analyzed by any immunodetection assay known to the skilled artisan. In some embodiments, an ELISA assay is used, while in others, the sample is assayed immunohistochemically.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-B A. Differential expression of total PSA (tPSA) in the 3 cell lines: MLC-SV40, LNCaP, C 4-2B. B. Differential expression of Free PSA (fPSA) in the 3 cell lines: MLC-SV40, LNCaP, C 4-2B.

FIG. 2 Dose-response kinetics of UC28 protein expression using Flow Cytometry and rabbit polyclonal antibody produced against UC28 and three prostate lines that differ in their malignant potential.

### DETAILED DESCRIPTION OF THE INVENTION

One of the most promising strategies for cancer therapy is induced cell differentiation. Cell differentiation is the process by which a daughter cell is different from its parent either through its cytoplasmic or its nuclear information. The changes are often expressed through turning genes on, and off and may be irreversible. If the run-away cell-division characteristic of cancer can be latched on with sufficient differentiation, then the accumulating changes will depress that lethal process of undifferentiated cell multiplication. Differentiation is an elegant response to cancer, it can handle small tumors as well as large ones, is not limited to particular types of cancer.

The present invention concerns methods and compositions for the diagnosis, prognosis targeting, treatment and prevention of cancer. It takes advantage of the observation that a differentiation agent can increase expression of UC28 or other UC markers in cancer cells, thus providing a way to target cancer cells. Compositions that target UC-28 or other UC marker-expressing cells are provided. In further detail below are various embodiments that also describe various other UC markers. “UC markers” refers to markers identified in U.S. Pat. No. 6,218,529, which is incorporated herein by reference; UC markers refers to the polypeptides that are encoded by the RNA transcripts differentially expressed in cancer cells compared to normal cells and that are identified by SEQ ID NO in U.S. Pat. No. 6,218,529. It is contemplated that all UC markers identified in U.S. patent may be U.S. Pat. No. 6,218,529 may be envisaged as targeting agents or targeted agents for immunotherapy. The invention further concerns modulators of UC markers. A “modulator” of a polypeptide is one that affects the following with respect to the polypeptide or any nucleic acid encoding the polypeptide: expression, half-life, turnover rate, localization, activity, function, stability, or folding.

### I. Proteinaceous Compositions

Proteinaceous compositions are involved in therapeutic, targeting, preventative and screening methods of the invention. Proteinaceous compositions, such as UC markers, can be the target of the therapeutic action of a targeting agent or moiety. In another embodiment of the invention, the proteinaceous composition may itself be the targeting agent that allows the targeting of the UC markers. Yet another embodiment of the invention contemplates the use of proteinaceous compositions as agents that enable or facilitate the targeting of UC markers. A “targeting agent” is defined as an agent or moiety that directs a compound to a particular composition or compound (target). For example, an anti-UC28 antibody is a targeting agent for UC28 (target) or a UC28-bearing cell. A “targeted agent” or “target” is an agent, compound, or moiety that is recognized by a targeting agent. These definitions will be used throughout the specification. The term “agent” is interchangeable with the term “moiety,” whenever appropriate. In embodiments of the present invention, a targeting agent is defined as an agent that targets UC marker proteins. A targeted agent is the UC marker protein itself, which ultimately allows cancer cells to be indirectly targeted. In some embodiments the UC marker can also act as a targeting agent.

In certain embodiments, the UC marker compositions of the invention are contemplated to be targeted moieties. The targeted moieties may be UC peptide or polypeptide sequences such as SEQ ID NO:2 or a fragment thereof, such as SEQ ID NO:3 or SEQ ID NO:4. U.S. Pat. No. 6,218,529 is specifically incorporated by reference. In that patent, the inventors have disclosed two alternate cDNA sequences for the UC28 gene corresponding to mRNA splice variants. One of them is designated as SEQ ID NO: 1 in the present application. Each sequence has the same open reading frame and encodes a protein with 135 amino acids. In the present application this UC28 amino acid sequence has been designated as SEQ ID NO: 2.

The present invention also contemplates the following peptide or polypeptide sequences: truncated neu; UC 38 or UC 41, UC31 and their fragments as targeted moieties. The SEQ ID NOs corresponding to nucleic acids encoding them are found later in the application.

As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

As mentioned above, the proteinaceous composition may also include targeting moieties and such molecules, in certain embodiments of the invention, may bear the size of at least one proteinaceous molecules that may comprise but is not limited to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 535 or greater amino molecule residues, and any range derivable therein. Such lengths are applicable to all peptides mentioned earlier in this section, including SEQ ID NO: 2, SEQ ID NO: 3 and SEQ ID NO: 4.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

The term “functionally equivalent codon” is used herein to refer to codons that encode the same amino acid, such as the six codons for arginine and serine, and also refers to codons that encode biologically equivalent amino acids. Codon usage for various organisms and organelles can be found at the website http://www.kazusa.or.ip/codon/, incorporated herein by reference, allowing one of skill in the art to optimize codon usage for expression in various organisms using the disclosures herein. Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria, chloroplasts and the like, based on the preferred codon usage as would be known to those of ordinary skill in the art.

It will also be understood that amino acid sequences or nucleic acid sequences of the targeted or targeting agents may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

Accordingly, the term “proteinaceous composition” encompasses amino molecule sequences comprising at least one of the 20 common amino acids in naturally synthesized proteins, or at least one modified or unusual amino acid, including but not limited to those shown on Table 1 below.

TABLE 1
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, β-Amino- | AHyl | allo-Hydroxylysine |
| | propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

In certain embodiments the proteinaceous composition of the targeting agents, targeted agents and those agents that allow the target to be targeted comprises at least one protein, polypeptide or peptide. In further embodiments the proteinaceous composition comprises a biocompatible protein, polypeptide or peptide. As used herein, the term “biocompatible” refers to a property of being biologically compatible thus producing no significant untoward effects when applied to, or administered to, a given organism according to the methods and amounts described herein. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

A. UC28 Protein, Polypeptides, and Peptides

The invention contemplates the use of differentiation agents, targeting agents, targeted agents, and inhibitors of UC marker polypeptide in the treatment of cancers. In some embodiments these may be targeted to a full-length or a substantially full-length UC polypeptide. The term “full-length” refers to a UC polypeptide such as UC28 that contains at least the 135 amino acids encoded by the UC28 cDNA. The term “substantially full-length” in the context of UC28 refers to a UC28 polypeptide that contains at least 80% of the contiguous amino acids of the full-length UC28 polypeptide. However, it is also contemplated that UC28 polypeptides containing at least about 85%, 90%, and 95% of SEQ ID NO:2 are within the scope of the invention as “substantially full-length” UC28. In other embodiments the UC28 polypeptide comprises at least 21 contiguous amino acid residues of SEQ ED NO:2 (For example, as SEQ NO 3). In still other aspects, the UC28 polypeptide comprises at least 17 contiguous amino acid residues of SEQ ID NO:2 (for example, SEQ ID NO:4).

The term “biologically functional equivalent” is well understood in the art and is further defined in detail herein. Accordingly, a sequence that has between about 70% and about 80%; or more preferably, between about 81% and about 90%; or even more preferably, between about 91% and about 99%; of amino acids that are identical or functionally equivalent to the amino acids such as SEQ ID NO:2 will be a sequence that is “essentially as set forth in SEQ NO:2,” provided the biological activity of the protein, polypeptide, or peptide is maintained.

The term “functionally equivalent codon” is used herein to refer to codons that encode the same amino acid, such as the six codons for arginine and serine, and also refers to codons that encode biologically equivalent amino acids (see Table 1).

Excepting intronic and flanlcing regions, and allowing for the degeneracy of the genetic code, nucleic acid sequences that have between about 70% and about 79%; or more preferably, between about 80% and about 89%; or even more particularly, between about 90% and about 99%; of nucleotides that are identical to the nucleotide such as SEQ ID NO:1.

It will also be understood, as mentioned earlier in the application, that this invention is not limited to the particular nucleic acid and amino acid sequences of SEQ NO:1 and SEQ ID NO:2 respectively.

Recombinant vectors and isolated nucleic acid segments may variously include the coding regions themselves, coding regions bearing selected alterations or modifications in the basic coding region, and they may encode larger polypeptides or peptides that nevertheless include such coding regions or may encode biologically functional equivalent proteins, polypeptide or peptides that have variant amino acids sequences.

The nucleic acids of the present invention encompass biologically functional equivalent UC28 proteins, polypeptides, or peptides. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Recombinant changes may be introduced, for example, through the application of site-directed mutagenesis techniques as discussed herein below, e.g., to introduce improvements or alterations to the antigenicity of the protein, polypeptide or peptide, or to test mutants in order to examine UC28 protein, polypeptide, or peptide activity at the molecular level.

In another embodiment of the invention, fusion proteins, polypeptides or peptides may be prepared, that are linked to an antibody that binds specifically to a UC marker. Non-limiting examples of such desired functions of expression sequences include purification or immunodetection purposes for the added expression sequences, e.g., proteinaceous compositions that may be purified by affinity chromatography or the enzyme labeling of coding regions, respectively.

The following is a discussion based upon changing of the amino acids of a protein, which in the present invention, may be the targeting agent or the targeted agent or an agent that allows the target to be targeted, to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, as discussed below. Table 1 shows the codons that encode particular amino acids.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *-0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing compounds, that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outlined above, to engineer second generation molecules having many of the natural properties of UC28 antigen or other UC marker antigens, but with altered and even improved characteristics. The same can be applied to UC antibodies or any other moiety that can serve as a targeting moiety.

B. Conjugates, Including Antibody Conjugates

The present invention further provides polypeptides, including antigens and antibodies against translated proteins, polypeptides and peptides, that may be linked to at least one agent to form a conjugate with some of the antibodies against UC marker proteins. In the present invention monoclonal antibodies have been prepared specifically to the following peptides: SEQ ID NO: 3 and SEQ ID NO: 4 to target UC 28 antigen. In order to increase the efficacy of proteinaceous molecules as screening, targeting or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules, which have been attached to antibodies, include toxins, anti-tumor agents, antibiotics, therapeutic enzymes, radio-labeled nucleotides, antiviral agents, chelating agents, cytokines, growth factors, and oligo- or poly-nucleotides. By contrast, a label or a detection agent is defined as any moiety that may be detected using an assay. Non-limiting examples of labels or detection reagents that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin. The examples that involve detection by color are generally understood to be colorimetric labels or detection reagents. Herein, “label” and “detection reagent” are used interchangeably.

Antibodies have been the main focus of protein conjugates and are discussed below. In the present invention, the antibody is a targeting agent against UC markers such as UC 28 marker antigen and all the antibody conjugates mentioned herein facilitate the targeting of the targeted moiety and hence the destruction of cancer cells that express this moiety. However, the examples of antibody conjugates may be applied more generally to any proteinaceous composition described herein.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins”.

Exemplary anticellular agents include chemotherapeutic agents, radioisotopes as well as cytotoxins. Example of chemotherapeutic agents are hormones such as steroids; antimetabolites such as cytosine arabinoside, fluorouracil, methotrexate or aminopterin; anthracycline; mitomycin C; vinca alkaloids; demecolcine; etoposide; mithramycin; or alkylating agents such as chlorambucil or melphalan.

Preferred immunotoxins often include a plant-, fungal- or bacterial-derived toxin, such as an A chain toxin, a ribosome inactivating protein, a-sarcin, aspergillin, restirictocin, a ribonuclease, diphtheria toxin or pseudomonas exotoxin. More particular examples include ribosome inhibitory proteins or apoptosis inducing agents. Ribosome inhibitory protein may be abrin, diphtheria toxin, gelonin, mitogillin, pseudomonas exotoxin, ricin A chain, saporin or shiga toxin. Apoptosis inducing agents may be BAD, Bax, TNFa, TNFβ, Fas-L, p-53, myc oncogene or onconase. Of course, combinations of the various toxins could also be coupled to one antibody molecule, thereby accommodating variable or even enhanced cytotoxicity.

One type of toxin for attachment to antibodies is ricin, with deglycosylated ricin A chain being particularly preferred. As used herein, the term “ricin” is intended to refer to ricin prepared from both natural sources and by recombinant means. Various ‘recombinant’ or ‘genetically engineered’ forms of the ricin molecule are known to those of skill in the art, all of which may be employed in accordance with the present invention.

Once conjugated, it will be important to purify the conjugate so as to remove contaminants such as unconjugated A chain or antibody. It is important to remove unconjugated A chain because of the possibility of increased toxicity. Moreover, it is important to remove unconjugated antibody to avoid the possibility of competition for the antigen between conjugated and unconjugated species. In any event, a number of purification techniques have been found to provide conjugates to a sufficient degree of purity to render them clinically useful.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (11), ytterbium (HI), gadolinium (III), vanadium (II), terbium (HI), dysprosium (III), holmium (HI) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus ¹⁸⁶, rhenium, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column.

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well Icnown to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another Ic.nown method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et at, 1985). The 2- and 8-azido nucleotides have also been used to map nucleotide binding domains of purified proteins (Khatoon et al., 1989; King et al., 1989; and Dholalcia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; ancUor tetrachloro-3a-6a-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

It also contemplated that conjugates may be multimeric. A polypeptide conjugate multimer refers to a proteinaceous compound that contains at least two amino acid regions, wherein the regions are from different organisms or polypeptides and wherein each region is attached to another region, covalently or non-covalently; this is described in U.S. Pat. No. 5,976,546, which is specifically incorporated by reference.

1. Linkers/Coupling Agents

If desired, compounds may be joined with other targeting compounds of the invention. A therapeutic, preventative, or targeting compound may be joined via a biologically-releasable bond, such as a selectively-cleavable linker or amino acid sequence. For example, peptide linkers that include a cleavage site for an enzyme preferentially located or active within a particular environment are contemplated. Exemplary forms of such peptide linkers are those that are cleaved by urokinase, plasmin, thrombin, Factor IXa, Factor Xa, or a metallaproteinase, such as collagenase, gelatinase, or stromelysin.

Amino acids such as selectively-cleavable linkers, synthetic linkers, or other amino acid sequences may be used to separate a compounds from one another.

Additionally, while numerous types of disulfide-bond containing linkers are known that can successfully be employed to conjugate compounds, such as an antibiotic to a polypeptide or a label to a polypeptide, certain linkers will generally be preferred over other linkers, based on differing pharmacologic characteristics and capabilities. For example, linkers that contain a disulfide bond that is sterically “hindered” are to be preferred, due to their greater stability in vivo, thus preventing release of the toxin moiety prior to binding at the site of action. Furthermore, certain advantages in accordance with the invention will be realized through the use of any of a number of toxin moieties. Details of the application of immunotoxins in the present invention are described elsewhere in the application.

Linking or coupling one or more toxin moieties to an antibody may be achieved by a variety of mechanisms, for example, covalent binding, affinity binding, intercalation, coordinate binding and complexation. Covalent binding methods use chemical cross-linkers, natural peptides or disulfide bonds. In the present invention, such compounds are linked to targeting agents such as antibodies against UC markers.

The covalent binding can be achieved either by direct condensation of existing side chains or by the incorporation of external bridging molecules. Many bivalent or polyvalent agents are useful in coupling protein molecules to other proteins, peptides or amine functions. Examples of coupling agents are carbodiimides, diisocyanates, glutaraldehyde, diazobenzenes, and hexamethylene diamines. This list is not intended to be exhaustive of the various coupling agents known in the art but, rather, is exemplary of the more common coupling agents that may be used.

2. Biochemical Cross-Linkers

The joining of any of the above components to targeting peptides will generally employ the same technology as developed for the preparation of immunotoxins. It can be considered as a general guideline that any biochemical cross-linker that is appropriate for use in an immunotoxin will also be of use in the present context, and additional linkers may also be considered.

Cross-linking reagents are used to form molecular bridges that tie together functional groups of two different molecules, e.g., a stablizing and coagulating agent. To link two different proteins in a step-wise manner, hetero-bifunctional cross-linkers can be used that eliminate unwanted homopolymer formation. Examples of hetero-bifunctional cross-linkers are presented in Table 2.

TABLE 2
HETERO-BIFUNCTIONAL CROSS-LINKERS

| | | | Spacer Arm Length\ |
|---|---|---|---|
| Linker | Reactive Toward | Advantages and Applications | after cross-linking |
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC-SPDP | Primary amines | Extended spacer arm | 15.6 A |
| | Sulfhydryls | Water-soluble | |
| SMCC | Primary amines | Stable maleimide reactive group | 11.6 A |
| | Sulfhydryls | Enzyme-antibody conjugation | |
| | | Hapten-carrier protein conjugation | |
| Sulfo-SMCC | Primary amines | Stable maleimide reactive group | 11.6 A |
| | Sulfhydryls | Water-soluble | |
| | | Enzyme-antibody conjugation | |
| MBS | Primary amines | Enzyme-antibody conjugation | 9.9 A |
| | Sulfhydryls | Hapten-carrier protein conjugation | |
| Sulfo-MBS | Primary amines | Water-soluble | 9.9 A |
| | Sulfhydryls | | |
| IAB | Primary amines | Enzyme-antibody conjugation | 10.6 A |
| | Sulfhydryls | | |
| Sulfo-SIAB | Primary amines | Water-soluble | 10.6 A |
| | Sulfhydryls | | |
| SMPB | Primary amines | Extended spacer arm | 14.5 A |
| | Sulfhydryls | Enzyme-antibody conjugation | |
| Sulfo-SMPB | Primary amines | Extended spacer arm | 14.5 A |
| | Sulfhydryls | Water-soluble | |
| EDC/Sulfo-NHS | Primary amines | Hapten-Carrier conjugation | 0 |
| | Carboxyl groups | | |
| ABH | Carbohydrates | Reacts with sugar groups | 11.9 A |
| | Nonselective | | |

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It can therefore be seen that a targeting peptide composition will generally have, or be derivatized to have, a functional group available for cross-linking purposes. This requirement is not considered to be limiting in that a wide variety of groups can be used in this manner. For example, primary or secondary amine groups, hydrazide or hydrazine groups, carboxyl alcohol, phosphate, or alkylating groups may be used for binding or cross-linking.

The spacer arm between the two reactive groups of cross-linkers may have various length and chemical compositions. A longer spacer arm allows a better flexibility of the conjugate components while some particular components in the bridge (e.g., benzene group) may lend extra stability to the reactive group or an increased resistance of the chemical link to the action of various aspects (e.g., disulfide bond resistant to reducing agents). The use of peptide spacers, such as L-Leu-L-Ala-L-Leu-L-Ala (SEQ ID NO:9), is also contemplated.

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagents for use in immunotoxins is SMPT, which is a bifunctional cross-linker containing a disulfide bond that is “sterically hindered” by an adjacent benzene ring and methyl groups. It is believed that stearic hindrance of the disulfide bond serves a function of protecting the bond from attack by thiolate anions such as glutathione which can be present in tissues and blood, and thereby help in preventing decoupling of the conjugate prior to the delivery of the attached agent to the tumor site. It is contemplated that the SMPT agent may also be used in connection with the bispecific coagulating ligands of this invention.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane. The use of such cross-linkers is well understood in the art.

Once conjugated, the polypeptide generally will be purified to separate the conjugated from unconjugated compounds and from other contaminants. A number of purification techniques are available for use in providing conjugates of a sufficient degree of purity to render them clinically useful. Purification methods based upon size separation, such as gel filtration, gel permeation or high performance liquid chromatography, will generally be of most use. Other chromatographic techniques, such as Blue-Sepharose separation, may also be used.

Blue-Sepharose is a column matrix composed of Cibacron Blue 3GA and agarose, which has been found to be useful in the purification of immunoconjugates. The use of Blue-Sepharose combines the properties of ion exchange with A chain binding to provide good separation of conjugated from unconjugated binding. The Blue-Sepharose allows the elimination of the free (non conjugated) antibody from the conjugate preparation. To eliminate the free (unconjugated) toxin (e.g., dgA) a molecular exclusion chromatography step may be used using either conventional gel filtration procedure or high performance liquid chromatography.

After a sufficiently purified conjugate has been prepared, one will generally desire to prepare it into a pharmaceutical composition that may be administered parenterally. This is done by using for the last purification step a medium with a suitable pharmaceutical composition. Such formulations will typically include pharmaceutical buffers, along with excipients, stabilizing agents and such like. The pharmaceutically acceptable compositions will be sterile, non-immunogenic and non-pyrogenic. Details of their preparation are well known in the art and are further described herein. It will be appreciated that endotoxin contamination should be kept minimally at a safe level, for example, less that 0.5 ng/mg protein.

Suitable pharmaceutical compositions in accordance with the invention will generally comprise from about 10 to about 100 mg of the desired conjugate admixed with an acceptable pharmaceutical diluent or excipient, such as a sterile aqueous solution, to give a final concentration of about 0.25 to about 2.5 mg/ml with respect to the conjugate.

In addition to chemical conjugation, a purified proteinaceous compound may be modified at the protein level. Included within the scope of the invention are protein fragments or other derivatives or analogs that are differentially modified during or after translation, for example by glycosylation, acetylation, phosphorylation, amidation, derivatization by known protecting/blocking groups, and proteolytic cleavage. Any number of chemical modifications may be carried out by known techniques, including but not limited to specific chemical cleavage by cyanogen bromide, trypsin, chymotrypsin, papain, V8 protease, NaBH₄; acetylation, formylation, farnesylation, oxidation, reduction; metabolic synthesis in the presence of tunicamycin.

It is contemplated that any proteinaceous conjugate discussed in this section may, if appropriate, be prepared recombinantly.

C. Chimeric Polypeptides and Proteins

In accordance with the objects of the present invention, a polynucleotide that encodes a chimeric protein, mutant polypeptide, biologically active fragment of chimeric protein, or functional equivalent thereof, may be used to generate recombinant DNA molecules that direct the expression of the chimeric protein, chimeric peptide fragments, or a functional equivalent thereof, in appropriate host cells. Such chimeric proteins may be used as targeting agents against the UC markers. A chimeric protein or polypeptide is characterized by an amino acid sequence not normally found in nature. Such a protein or Polypeptide generally has an amino acid sequence from more than one protein or polypeptide or from the same protein or polypeptide but from a different species of organism. A chimeric protein may have sequences from one polypeptide inserted into a second polypeptide recombinantly.

D. Fusion Proteins

A specialized lcind of insertional variant of a chimeric protein is the fusion protein. Fusion proteins are contemplated as part of the invention. In the present invention these may be linked to a targeting agent against UC markers. In some embodiments a UC protein or polypeptide or a fragment thereof may be linked at the N- or C-terminus to another polypeptide or its fragment. General examples include fusions that typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions. Additionally, a proteinaceous label may be placed onto the end of a polypeptide. Fusions may be generated recombinantly, as distinguished from protein conjugates, which are chemically generated. The use of recombinant DNA techniques to achieve such ends is now standard practice to those of slcill in the art. These methods include, for example, in vitro recombinant DNA techniques, synthetic techniques and in vivo recombination/genetic recombination. DNA and RNA synthesis may, additionally, be performed using an automated synthesizers (see, for example, the techniques described in Sambrook et al., 1989; and Ausubel et al., 1989).

The preparation of such a fusion protein generally entails the preparation of a first and second DNA coding region and the functional ligation or joining of said regions, in frame, to prepare a single coding region that encodes the desired fusion protein.

Once the coding region desired has been produced, an expression vector is created. Expression vectors contain one or more promoters upstream of the inserted DNA regions that act to promote transcription of the DNA and to thus promote expression of the encoded recombinant protein. This is the meaning of “recombinant expression” and has been discussed elsewhere in the specification.

Fusion proteins, polypeptides or peptides may be prepared, e.g., where the coding regions are aligned within the same expression unit with other proteins, polypeptides or peptides having desired functions. Non-limiting examples of such desired functions of expression sequences include purification or immunodetection purposes for the added expression sequences, e.g., proteinaceous compositions that may be purified by affinity chromatography or the enzyme labeling of coding regions, respectively.

E. Use of Peptide Mimetics

Another method for the preparation of the polypeptides according to the invention is the use of peptide mimetics. In the present embodiment of the invention it is contemplated that such polypeptides mimic elements of UC markers and may thus be used as vaccines. Mimetics are peptide-containing compounds, which mimic elements of protein secondary structure. See, for example, Johnson et al., “Peptide Turn Mimetics” in BIOTECHNOLOGY AND PHARMACY, Pezzuto et al., Eds., Chapman and Hall, New York (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule.

Successful applications of the peptide mimetic concept have thus far focused on mimetics of β-turns within proteins, which are known to be highly antigenic. Likely 13-turn structure within a polypeptide may be predicted by computer-based algorithms as discussed herein. Once the component amino acids of the turn are determined, peptide mimetics may be constructed to achieve a similar spatial orientation of the essential elements of the amino acid side chains.

F. Purification of Polypeptides

Further aspects of the present invention concern the purification, and in particular embodiments, the substantial purification of an encoded protein or peptide that serve as a targeting agent. In some aspects it deals with the purification of the targeted agent. In some aspects it also deals with the purification of the targeted agent. The term “purified protein or peptide” as used herein, is intended to refer to a composition, isolatable from other components, wherein the protein or peptide is purified to any degree relative to its naturally-obtainable state, i.e., in this case, relative to its purity within a prostate, bladder or breast cell extract. A purified protein or peptide therefore also refers to a protein or peptide, free from the environment in which it may naturally occur.

Generally, “purified” will refer to a protein or peptide composition which has been subjected to fractionation to remove various other components, and which composition substantially retains its expressed biological activity. Where the term “substantially purified” is used, this will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number”. The actual units used to represent the amount of activity will, of course, be dependent upon the particular assay technique chosen to follow the purification and whether or not the expressed protein or peptide exhibits a detectable activity.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the cohunn, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand also should provide relatively tight binding. And it should be possible to elute the substance without destroying the sample or the ligand. One of the most common forms of affinity chromatography is immunoaffinity chromatography.

G. Antibody Generation

For some embodiments, the targeting agent may be an antibody specific to a UC 28 protein marker or any other UC marker, such as UC 31; truncated neu; UC 38; UC 41. Means for preparing and characterizing antibodies are well known in the art (See, e.g., Antibodies: A Laboratory Manual, Cold Spring Harbor Laboratory, 1988; incorporated herein by reference). Antibodies may also be used or prepared as discussed.

1. Polyclonal Antibodies

Methods for generating polyclonal antibodies are well known in the art. Briefly, a polyclonal antibody is prepared by immunizing an animal with an immunogenic composition and collecting antisera from that immunized animal. A wide range of animal species may be used for the production of antisera. Typically the animal used for production of anti-antisera is a rabbit, a mouse, a rat, a hamster, a guinea pig or a goat. Because of the relatively large blood volume of rabbits, a rabbit is a preferred choice for production of polyclonal antibodies. In the present invention rabbit polyclonal antibodies to UC 28 peptides SEQ ID NO:3 and SEQ ID NO:4 have been prepared and are designated as UC28A 1 and UC28C1.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin may also be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As is also well known in the art, the immunogenicity of a particular immunogen composition may be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes may be used to administer the immunogen (subcutaneous, intramuscular, intradermal, intravenous and intraperitoneal). The production of polyclonal antibodies may be monitored by sampling blood of the immunized animal at various points following immunization. A second, booster injection, may also be given. The process of boosting and titering is repeated until a suitable titer is achieved. When a desired level of immunogenicity is obtained, the immunized animal may be bled and the serum isolated and stored, and/or the animal may be used to generate MAbs. For production of rabbit polyclonal antibodies, the animal may be bled through an ear vein or alternatively by cardiac puncture. The removed blood is allowed to coagulate and then centrifuged to separate senun components from whole cells and blood clots. The serum may be used as is for various applications or else the desired antibody fraction may be purified by well-known methods, such as affinity chromatography using another antibody or a peptide bound to a solid matrix.

2. Monoclonal Antibodies (MAbs)

Monoclonal antibodies (MAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed protein, polypeptide or peptide. The immunizing composition is administered in a manner effective to stimulate antibody producing cells. In the present embodiment of the invention monoclonal antibodies have been prepared to UC 28 peptides SEQ ID NO: 3 and SEQ ID NO:4. These monoclonal antibodies have been designated as UC28A 3-1 G2, UC28A 1-4 A3, UC28A 3-3 G10, UC28A 1-4 C9, UC28A 4-1 H5, UC28C 2-2 D2, UC28C 1-1 A1, UC28C 1-1 A2, UC 28C 3-1F3 or UC 28C 2-3 G2.

The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fizions.

The animals are injected with antigen as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

In accordance with the present invention, fragments of the monoclonal antibody of the invention may be obtained from the monoclonal antibody produced as described above, by methods which include digestion with enzymes such as pepsin or papain and/or cleavage of disulfide bonds by chemical reduction. Alternatively, monoclonal antibody fragments encompassed by the present invention may be synthesized using an automated peptide synthesizer.

The monoclonal conjugates of the present invention are prepared by methods known in the art, e.g., by reacting a monoclonal antibody prepared as described above with, for instance, an enzyme in the presence of a coupling agent such as glutaraldehyde or periodate. Conjugates with fluorescein markers are prepared in the presence of these coupling agents or by reaction with an isothiocyanate. Conjugates with metal chelates are similarly produced. Other moieties to which antibodies may be conjugated include radionuclides such as ³H, ¹²⁵I, ¹³¹I, ³²P, ³⁵S, ¹⁴C, ^{5I}Cr, ³⁶Cl, ⁵⁷Co, ⁵⁸Co, ⁵⁹Fe, ⁷⁵Se, ¹⁵²Eu, and ^{99m}Tc. As mentioned earlier in the description radioactively labeled monoclonal antibodies of the present invention are produced according to well-known methods in the art.

3. Humanized Antibodies

Humanized monoclonal antibodies are antibodies of animal origin that have been modified using genetic engineering techniques to replace constant region and/or variable region framework sequences with human sequences, while retaining the original antigen specificity. Such antibodies are commonly derived from rodent antibodies with specificity against human antigens. Such antibodies are generally useful for in vivo therapeutic applications. This strategy reduces the host response to the foreign antibody and allows selection of the human effector functions.

“Humanized” antibodies are also contemplated, as are chimeric antibodies from mouse, rat, or other species, bearing human constant and/or variable region domains, bispecific antibodies, recombinant and engineered antibodies and fragments thereof. The techniques for producing humanized immunoglobulins are well known to those of skill in the art. For example U.S. Pat. No. 5,693,762 discloses methods for producing, and compositions of, humanized immunoglobulins having one or more complementarity determining regions (CDR's). When combined into an intact antibody, the humanized immunoglobulins are substantially non-immunogenic in humans and retain substantially the same affinity as the donor immunoglobulin to the antigen, such as a protein or other compound containing an epitope. Examples of other teachings in this area include U.S. Pat. Nos. 6,054,297; 5,861,155; and 6,020,192, all specifically incorporated by reference. Methods for the development of antibodies that are “custom-tailored” to the patient's disease are likewise known and such custom-tailored antibodies are also contemplated.

H. Immunotoxins

The invention further provides a type of fusion or chimeric polypeptide such as an immunotoxin. In the present invention, immunotoxins may be linked to a targeting agent or moiety.

Immunotoxin technology is fairly well-advanced and known to those of skill in the art. Immunotoxins are agents in which the antibody component is linked to another agent, particularly a cytotoxic or otherwise anticellular agent, having the ability to kill or suppress the growth or cell division of cells.

As used herein, the terms “toxin” and “toxic moiety” are employed to refer to any cytotoxic or otherwise anticellular agent that has such a killing or suppressive property. Toxins are thus pharmacologic agents that can be conjugated to an antibody and delivered in an active form to a cell, wherein they will exert a significant deleterious effect.

The preparation of immunotoxins is, in general, well known in the art (see, e.g., U.S. Pat. No. 4,340,535, incorporated herein by reference). The toxins of the invention are also suited for use as components of cytotoxic therapeutic agents. These cytotoxic agents may be used in vivo to selectively eliminate a particular cell type to which the toxin component is targeted by the specific binding capacity of a second component. To form cytotoxic agents, modified toxins of the present invention may be conjugated to monoclonal antibodies, including chimeric and CDR-grafted antibodies, and antibody domains/fragments (e.g., Fab, Fab′, F(ab′).sub.2, single chain antibodies, and Fv or single variable domains).

Immunoconjugates including toxins may be described as immunotoxins. An immunotoxin may also consist of a fusion protein (recombinant) rather than an immunoconjugate.

Modified toxins conjugated to monoclonal antibodies genetically engineered to include free cysteine residues are also within the scope of the present invention. Examples of Fab′ and F(ab′).sub.2 fragments useful in the present invention are described in WO 89/00999, which is incorporated by reference herein.

Alternatively, the modified toxins may be conjugated or fused to humanized or human engineered antibodies. Such humanized antibodies may be constructed from mouse antibody variable domains. Humanized antibodies have been described above.

1. Antibody Regions

Regions from the various members of the immunoglobulin family are encompassed by the present invention. Both variable regions from specific antibodies are covered within the present invention, including complementarity determining regions (CDRs), as are antibody neutralizing regions, including those that bind effector molecules such as Fc regions. Antigen specific-encoding regions from antibodies, such as variable regions from IgGs, IgMs, or IgAs, can be employed with a UC marker-binding domain in combination with an antibody neutralization region or with one of the therapeutic compounds described above. It also is known that while IgG based immunotoxins will typically exhibit better binding capability and slower blood clearance than their Fab′ counterparts, Fab′ fragment-based immunotoxins will generally exhibit better tissue penetrating capability as compared to IgG based immunotoxins.

In yet another embodiment, one gene may comprise a single-chain antibody. Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen binding site on a single molecule.

Single-chain antibody variable fragments (scFvs) in which the C-terminus of one variable domain is tethered to the N-terminus of the other via a 15 to 25 amino acid peptide or linker, have been developed without significantly disrupting antigen binding or specificity of the binding (Bedzyk et al., 1990; Chaudhary et al., 1990). These Fvs lack the constant regions (Fc) present in the heavy and light chains of the native antibody. Immunotoxins employing single-chain antibodies are described in U.S. Pat. No. 6,099,842, specifically incorporated by reference.

Antibodies to a wide variety of molecules are contemplated, such as oncogenes, tumor-associated antigens, cytokines, growth factors, hormones, enzymes, transcription factors or receptors. Also contemplated are secreted antibodies targeted against serum, angiogenic factors (VEGFNPF; βFGF; αFGF; and others), coagulation factors, and endothelial antigens necessary for angiogenesis (i.e., V3 integrin). Specifically contemplated are growth factors such as transforming growth factor, fibroblast growth factor, and platelet derived growth factor (PDGF) and PDGF family members.

The antibodies employed in the present invention as part of an immunotoxin may be targeted to any antigen. The antigen may be specific to an organism, to a cell type, to a disease or condition, or to a pathogen. Exemplary antigens include cell surface cellular proteins, for example tumor-associated antigens, viral proteins, microbial proteins, post-translational modifications or carbohydrates, and receptors. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

2. Other Targeting Moieties

The use of a region of a protein that mediates protein-protein interactions, including ligand-receptor interactions, also is contemplated by the present invention. Specifically included are moieties that would allow targeting the polypeptides involved in cancer cells compared to non-cancer cells. For example, other than anti-UC 28 antibodies, other moieties that specifically bind or target UC markers discussed throughout are contemplated. This region could be used as an inhibitor or competitor of a protein-protein interaction or as a specific targeting motif. Consequently, the invention covers using the targeting moiety to recruit the toxin or other therapeutic or diagnostic polypeptide to a particular body part, organ, tissue, or cell. Once the compositions of the present invention reach the particular area through the targeting motif, the toxin or other polypeptide can function.

Targeting moieties may take advantage of protein-protein interactions. These include interactions between and among proteins such as receptors and ligands; receptors and receptors; polymeric complexes; transcription factors; kinases and downstream targets; enzymes and substrates; etc. For example, a ligand binding domain mediates the protein:protein interaction between a ligand and its cognate receptor. Consequently, this domain could be used either to inhibit or compete with endogenous ligand binding or to target more specifically cell types that express a receptor that recognizes the ligand binding domain operatively attached to a therapeutic polypeptide, such as the gelonin toxin.

I. Targeted Inhibition of UC Cancer Markers

This section further concerns with other targeting agents that inhibit UC markers. The present invention concerns particular amino acid sequences that can be employed for targeting cancerous cells and not normal cells.

One of the identified genes, cyclin A, has been described as a target for a number of agents that inhibit tumor cell growth by promoting differentiation or inhibiting cell division. For example, L-tyrosine has been reported to promote increased melanogenesis and replicative senescence in the B16 melanoma cell line, correlated with a decrease in cyclin A activity. (Rieber and Rieber, 1994) Suramin is an antitumor agent that reduces the expression of cyclin A in the DU-145 prostate carcinoma cell line. (Qiao et al., 1994) Rapamycin inhibits cell proliferation in the YAC-1 T cell lymphoma and also inhibits cyclin A mRNA production (Dumont et al., 1994) It is not clear if these inhibitors are acting directly on cyclin A, or somewhere upstream in a signal transduction/phosphorylation cascade pathway. However, inhibitors of cyclin A should inhibit cell proliferation and decrease tumor growth. Such inhibitors may have utility as therapeutic agents for the treatment of cancer.

Identification of protein function may be extrapolated, in some cases, from the primary sequence data, provided that sequence homology exists between the unknown protein and a protein of similar sequence and known function. Proteins tend to occur in large families of relatively similar sequence and function. For example, a number of the serine proteases, like trypsin and chymotrypsin, have extensive sequence homologies and relatively similar three-dimensional structures. Other general categories of homologous proteins include different classes of transcriptional factors, membrane receptor proteins, tyrosine kinases, GTP-binding proteins, etc. The putative amino acid sequences encoded by the cancer-marker nucleic acids of the present invention may be cross-checked for sequence homologies versus the protein sequence database of the National Biomedical Research Fund. Homology searches are standard techniques for the skilled practitioner.

Even three-dimensional structure may be inferred from the primary sequence data of the encoded proteins. Again, if homologies exist between the encoded amino acid sequences and other proteins of known structure, then a model for the structure of the encoded protein may be designed, based upon the structure of the known protein. An example of this type of approach was reported by Ribas de Pouplana and Fothergill-Gilmore. These authors developed a detailed three-dimensional model for the structure of Drosophila alcohol dehydrogenase, based in part upon sequence homology with the known structure of 3-α, 20-β-hydroxysteroid dehydrogenase. Once a three-dimensional model is available, inhibitors may be designed by standard computer modeling techniques. This area has been recently reviewed by Sun and Cohen (1993), herein incorporated by reference.

### II. Immunodetection Assays

In still further embodiments, the present invention concerns immunodetection methods for binding, purifying, removing, quantifying or otherwise generally detecting biological components. The present invention contemplates the use of the UC markers as vaccines for the prevention of cancer. Such immunodetection methods may be involved in detecting the efficacy of the vaccine when administered to a cell or a patient. Further, immunodetection is also useful in detecting the upregulation of expression of UC 28 before or after the administration of a therapeutic agent or a differentiation agent. In the present embodiment of the invention immunological methods are applicable in detecting all proteinaceous compositions that may be used as targeting agents or that may be linked to the targeting agent as described earlier in the application. Also the immunodetection methods may also be used to quantify the antigen antibody complexes formed when anti-UC marker antibodies are administered to a patient having cancer. The steps of various useful immunodetection methods have been described in the scientific literature, such as, e.g., Nakamura et al. (1987).

In general, the immunobinding methods include obtaining a sample suspected of containing a protein, peptide or antibody, and contacting the sample with an antibody or protein or peptide in accordance with the present invention, as the case may be, under conditions effective to allow the formation of immunocomplexes.

The immunobinding methods include methods for detecting or quantifying the amount of a reactive component in a sample, which methods require the detection or quantitation of any immune complexes formed during the binding process. Here, one would obtain a sample suspected of containing a UC cancer marker encoded protein, peptide or a corresponding antibody, and contact the sample with an antibody or encoded protein or peptide, as the case may be, and then detect or quantify the amount of immune complexes formed under the specific conditions.

Contacting the chosen biological sample with the protein, peptide or antibody under conditions effective and for a period of time sufficient to allow the formation of immune complexes (primary immune complexes) is generally a matter of simply adding the composition to the sample and incubating the mixture for a period of time long enough for the antibodies to form immune complexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The encoded protein, peptide or corresponding antibody employed in the detection may itself be linked to a detectable label, wherein one would then simply detect this label, thereby allowing the amount of the primary immune complexes in the composition to be determined.

Alternatively, the first added component that becomes bound within the primary immune complexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the encoded protein, peptide or corresponding antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

A. Immunohistochemistry

Immunohistochemical methods may be used for all applications of immunodetection methods in the present invention as described in the previous section. The antibodies of the present invention may be used in conjunction with both fresh-frozen and formalin-fixed, paraffin-embedded tissue blocks prepared by immunohistochemistry (IHC). Any IHC method well known in the art may be used such as those described in particular, Chapter 31 of that reference entitled Gynecological and Genitourinary Tumors (pages 579-597), by Debra A. Bell, Robert H. Young and Robert E. Scully and references therein.

B. ELISA

As noted, it is contemplated that the encoded proteins or peptides of the invention will find utility as immunogens, e.g., in connection with vaccine development, in immunohistochemistry and in ELISA assays. One evident utility of the encoded antigens and corresponding antibodies is in immunoassays for the detection of cancer marker proteins, as needed in detection, prevention, therapeutics, diagnostics and prognostics of cancer.

Immunoassays, in their most simple and direct sense, are binding assays. Certain preferred immunoassays are the various types of enzyme linked immunosorbent assays (ELISAs) and radioimmunoassays (RIA) known in the art. Immunohistochemica] detection using tissue sections is also particularly useful. However, it will be readily appreciated that detection is not limited to such techniques, and Western blotting, dot blotting, FACS analyses, and the like may also be used.

In one exemplary ELISA, antibodies binding to the encoded proteins of the invention are immobilized onto a selected surface exhibiting protein affinity, such as a well in a polystyrene microliter plate. Then, a test composition suspected of containing the cancer marker antigen, such as a clinical sample, is added to the wells. After binding and washing to remove non-specifically bound immunecomplexes, the bound antigen may be detected. Detection is generally achieved by the addition of a second antibody specific for the target protein, that is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

In another exemplary ELISA, the samples suspected of containing the cancer marker antigen are immobilized onto the well surface and then contacted with the antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Another ELISA in which the proteins or peptides are immobilized, involves the use of antibody competition in the detection. In this ELISA, labeled antibodies are added to the wells, allowed to bind to the cancer marker protein, and detected by means of their label. The amount of marker antigen in an unknown sample is then determined by mixing the sample with the labeled antibodies before or during incubation with coated wells. The presence of marker antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immunecomplexes. These are described as follows:

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine senun albiunin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a se,condary or tertiary detection means rather than a direct procedure. Thus, after binding of a protein or antibody to the well, coating with a non-reactive material to reduce backgr, ound, and washing to remove unbound material, the immobilizing surface is contacted with the control human cancer ancUor clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

“Under conditions effective to allow immunecomplex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours, at temperatures preferably on the order of 25° to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immunecomplex with a unease, glucose oxidase, alkaline phosphatase or hydrogen peroxidase-conjugated antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂0₂, in the case of peroxidase as the enzyme label. Quantitation is then achieved by measuring the degree of color generation, e.g., using a visible spectra spectrophotometer.

C. Use of Antibodies for Radioimaging

The antibodies of this invention will be used to quantify and localize the expression of the encoded marker proteins. The antibody, for example, will be labeled by any one of a variety of methods and used to visualize the localized concentration of the cells producing the encoded protein.

The invention also relates to an in vivo method of imaging a cancer condition using the above described monoclonal antibodies. Specifically, this method involves administering to a subject an imaging-effective amount of a detectably-labeled cancer-specific monoclonal antibody or fragment thereof and a pharmaceutically effective carrier and detecting the binding of the labeled monoclonal antibody to the diseased tissue. The term “in vivo imaging” refers to any method which permits the detection of a labeled monoclonal antibody of the present invention or fragment thereof that specifically binds to a diseased tissue located in the subject's body. A “subject” is .a mammal, preferably a human. An “imaging effective amount” means that the amount of the detectablylabeled monoclonal antibody, or fragment thereof, administered is sufficient to enable detection of binding of the monoclonal antibody or fragment thereof to the diseased tissue.

A factor to consider in selecting a radionuclide for in vivo diagnosis is that the half-life of a nuclide be long enough so that it is still detectable at the time of maximum uptake by the target, but short enough so that deleterious radiation upon the host, as well as background, is minimized. Ideally, a radionuclide used for in vivo imaging will lack a particulate emission, but produce a large number of photons in a 140-2000 keV range, which may be readily detected by conventional gamma cameras.

A radionuclide may be bound to an antibody either directly or indirectly by using an intermediary functional group. Intermediary functional groups which are often used to bind radioisotopes which exist as metallic ions to antibody are diethylenetriaminepentaacetic acid (DTPA) and ethylene diaminetetracetic acid (EDTA). Examples of metallic ions suitable for use in this invention are ^{99m}Tc, ¹²³I, ¹³¹I, ¹¹¹In, ⁹⁷Ru, ⁶⁷Cu, ⁶⁷Ga, ¹²⁵I, ⁶⁸Ga, ⁷²As, ⁸⁹Zr, and ²⁰¹Tl. In accordance with this invention, the monoclonal antibody or fragment thereof may be labeled by any of several techniques known to the art. The methods of the present invention may also use paramagnetic isotopes for purposes of in vivo detection. Elements particularly useful in Magnetic Resonance Imaging (“MRI”) include ¹⁵⁷Gd, ⁵⁵Mn, ¹⁶²Dy, ⁵²Cr, and ⁵⁶Fe.

Administration of the labeled antibody may be local or systemic and accomplished intravenously, intraarterially, via the spinal fluid or the like. Administration may also be intradermal or intracavitary, depending upon the body site under examination. After a sufficient time has lapsed for the monoclonal antibody or fragment thereof to bind with the diseased tissue, for example 30 minutes to 48 hours, the area of the subject under investigation is examined by routine imaging techniques such as MRI, SPECT, planar scintillation imaging and emerging imaging techniques, as well. The exact protocol will necessarily vary depending upon factors specific to the patient, as noted above, and depending upon the body site under examination, method of administration and type of label used; the determination of specific procedures would be routine to the skilled artisan. The distribution of the bound radioactive isotope and its increase or decrease with time is then monitored and recorded. By comparing the results with data obtained from studies of clinically normal individuals, the presence and extent of the diseased tissue may be determined.

It will be apparent to those of skill in the art that a similar approach may be used to radio-image the production of the encoded cancer marker proteins in human patients. The present invention provides methods for the in vivo monitoring the course of treatment of cancer in a patient. Such methods generally comprise administering to a patient an effective amount of a cancer specific antibody, which antibody is conjugated to a marker, such as a radioactive isotope or a spin-labeled molecule, that is detectable by non-invasive methods. The antibody-marker conjugate is allowed sufficient time to come into contact with reactive antigens that are present within the tissues of the patient, and the patient is then exposed to a detection device to identify the detectable marker. It also allows for the monitoring of levels of upregulation of cancer marker antigens before or after a differentiation agent is administered to the patient.

D. FACS Analyses

Fluorescent activated cell sorting, flow cytometry or flow microfluorometry provides the means of scanning individual cells for the presence of an antigen. The method employs instrumentation that is capable of activating, and detecting the excitation emissions of labeled cells in a liquid medium. FAGS may be used before or after administration of the differentiation agent. It may also be used before or after the administration of a anti-UC-antibody.

FAGS is unique in its ability to provide a rapid, reliable, quantitative, and multiparameter analysis on either living or fixed cells. The cancer antibodies of the present invention provide a useful tool for the analysis and quantitation of antigenic cancer markers of individual cells.

Cells would generally be obtained by biopsy, single cell suspension in blood or culture. FAGS analyses would probably be most useful when desiring to analyze a number of cancer antigens at a given time, e.g., to follow an antigen profile during disease progression.

### III. Nucleic.Acids

The present invention contemplates the use of a variety of proteinaceous compositions, and accordingly, nucleic acids encoding such compositions are contemplated in the present invention. Furthermore, nucleic acids may be employed as modulators of UC markers, such as antisense or ribozyme molecules. In some embodiments of the invention these markers themselves may be an object of the targeting therapy. The proteins and polypeptides that are described above are encoded by the nucleic acids whose SEQ LD NOs are listed below.

| SEQ ID NO | Protein |
|---|---|
| 1. | UC28 |
| 5. | Truncated NEU |
| 6. | UC 38 |
| 7. | UC 41 |
| 8. | UC 31 |

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

A. Preparation of Nucleic Acids

The nucleic acids as listed above may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by U.S. Pat. No. 5,705,629, incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

B. Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference).

C. Nucleic Acid Segments

In certain embodiments, the nucleic acid is a nucleic acid segment, such as one encoding a proteinaceous composition described earlier in the application. As used herein, the term “nucleic acid segment,” are smaller fragments of a nucleic acid, such as for non-limiting example, those that encode ‘only part of the protein. Thus, a “nucleic acid segment” may comprise any part of a gene sequence, of from about 2 nucleotides to the full length of the protein.

In a non-limiting example, nucleic acid segments encoding a portion of the proteinaceous composition as described earlier such as UC marker protein, antibodies to UC marker protein, antibody conjugates, linIcers etc. may comprise or be limited to 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, or 5000 nucleotides such segements lengths may be applied with respect to SEQ NO: 1, SEQ ID NO: 5, SEQ NO: 6, SEQ NO: 7 and SEQ ID NO: 8., for example, or may include such segments of contiguous nucleotides from SEQ NO: 1, SEQ NO: 5, SEQ ID NO: 6, SEQ NO: 7 and SEQ NO: 8.

As used herein “wild-type” refers to the naturally occurring sequence of a nucleic acid at a genetic locus in the genome of an organism, or a sequence transcribed or translated from such a nucleic acid. Thus, the term “wild-type” also may refer to an amino acid sequence encoded by a nucleic acid. As a genetic locus may have more than one sequence or alleles in a population of individuals, the term “wild-type” encompasses all such naturally occurring allele(s). As used herein the term “polymorphic” means that variation exists (i.e., two or more alleles exist) at a genetic locus in the individuals of a population. As used herein “mutant” refers to a change in the sequence of a nucleic acid or its encoded protein, polypeptide or peptide that is the result of the hand of man.

The present invention also concerns the isolation or creation of a recombinant construct or a recombinant host cell through the application of recombinant nucleic acid technology known to those of skill in the art or as described herein. A recombinant construct or host cell may express any one of the proteinaceous compositions described before or at least one biologically functional equivalent thereof. The recombinant host cell may be a prokaryotic cell. In a more preferred embodiment, the recombinant host cell is a eukaryotic cell. As used herein, the term “engineered” or “recombinant” cell is intended to refer to a cell into which a recombinant gene, such as a gene encoding a protein described earlier, has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a cDNA gene (i.e., they will not contain introns), a copy of a genomic gene, or will include genes positioned adjacent to a promoter not naturally associated with the particular introduced gene.

Herein certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide, termed “coding sequence.” As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). As used herein, a “nucleic acid construct” is a nucleic acid engineered or altered by the hand of man, and generally comprises one or more nucleic acid sequences organized by the hand of man.

In a non-limiting example, one or more nucleic acid constructs may be prepared containing 3, 5, 8, 10 to 14, or 15, 20, 30, 40, 50, 100, 200, 500, 1,000, 2,000, 3,000, 5,000, 10,000, 15,000, 20,000, 30,000, 50,000, 100,000, 250,000 500,000, 750,000, to 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include 11, 12, 13, 16, 17, 18, 19, 20; 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 101, 102, 103; 151, 152, 153, 1,001, 1002; 50,001, 50,002, etc; about 750,001, about 750,002, etc.; about 1,000,0W,1,000,002, etc. Non-limiting examples of intermediate ranges include about 3 to about 32, about 150 to about 500,001, about 3,032 to about 7,145, about 5,000 to about 15,000, about 20,007 to about 1,000,003, etc.

The nucleic acids of the present invention encompass biologically functional equivalent UC proteins, polypeptides, or peptides or other proteinaceous compositions. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements or alterations to the antigenicity of the protein, polypeptide or peptide, or to test mutants in order to examine the UC protein, polypeptide or peptide activity at the molecular level.

As used herein an “organism” may be a prokaryote, eukaryote, virus and the like. As used herein the term “sequence” encompasses both the terms “nucleic acid” and “proteinaceous” or “proteinaceous composition.” As used herein, the term “proteinaceous composition” encompasses the terms “protein”, “polypeptide” and “peptide.” As used herein “artificial sequence” refers to a sequence of a nucleic acid not derived from sequence naturally occurring at a genetic locus, as well as the sequence of any proteins, polypeptides or peptides encoded by such a nucleic acid. A “synthetic sequence”, refers to a nucleic acid or proteinaceous composition produced by chemical synthesis in vitro, rather than enzymatic production in vitro (i.e., an “enzymatically produced” sequence) or biological production in vivo (i.e., a “biologically produced” sequence).

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE'S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REx™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

The nucleotide and protein, polypeptide and peptide sequences for various UC marker expressing genes have been previously disclosed (incorporated by Reference herein is U.S. Pat. No. 6,218,529) and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or by any technique that would be known to those of ordinary skill in the art. Additionally, peptide sequences may be synthesized by methods known to those of ordinary skill in the art, such as peptide synthesis using automated peptide synthesis machines, such as those available from Applied Biosystems (Foster City, Calif.).

Certain embodiments of the present invention involve the synthesis, creation, and/or mutation of a nucleic acid molecule and recombinant vectors encoding one or more UC proteins or any other proteinaceous compositions described earlier in the application. Thus, a mutation may be introduced in the gene encoding the protein. Embodiments of the invention also involve the creation and use of recombinant host cells through the application of DNA technology, that express one or more of the proteinaceous compounds described herein. In certain aspects, a nucleic acid encoding a protein or polypeptide comprises a wild-type or a mutant nucleic acid.

In one embodiment, the nucleic acid sequences encoding the UC marker proteins will find utility as hybridization probes. These nucleic acids may be used, for example, in diagnostic evaluation of tissue samples or employed to clone fill' length cDNAs or genomic clones corresponding thereto. In certain embodiments, these probes consist of oligonucleotide fragments. Such fragments should be of sufficient length to provide specific hybridization to a RNA or DNA tissue sample. The sequences typically will be 10-20 nucleotides, but may be longer. Longer sequences, e.g., 40, 50, 100, 500 and even up to full length, are preferred for certain embodiments.

Various probes can be designed around the above nucleotide sequences encoding the UC marker. The use of a hybridization probe of between 14 and 100 nucleotides in length allows the formation of a duplex molecule that is both stable and selective. Molecules having complementary sequences over stretches greater than 20 bases in length are generally preferred, in order to increase stability and selectivity of the hybrid, and thereby improve the quality and degree of particular hybrid molecules obtained. One will generally prefer to design nucleic acid molecules having stretches of 20 to 30 nucleotides, or even longer where desired. Such fragments may be readily prepared by, for example, directly synthesizing the fragment by chemical means or by introducing selected sequences into recombinant vectors for recombinant production.

Accordingly, the nucleotide sequences of the invention may be used for their ability to selectively form duplex molecules with complementary stretches of genes or RNAs or to provide primers for amplification of DNA or RNA from tissues. Depending on the application envisioned, one will desire to employ varying conditions of hybridization to achieve varying degrees of selectivity of probe towards target sequence.

For applications requiring high selectivity, one will typically desire to employ relatively stringent conditions to form the hybrids, e.g., one will select relatively low salt and/or high temperature conditions, such as provided by about 0.02 M to about 0.10 M NaCl at temperatures of about 50° C. to about 70° C. Such high stringency conditions tolerate little, if any, mismatch between the probe and the template or target strand, and would be particularly suitable for isolating specific genes or detecting specific in RNA transcripts. It is generally appreciated that conditions can be rendered more stringent by the addition of increasing amounts of formamide.

For certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

The codon chart in Table 3 may be used, in a site-directed mutagenic scheme, to produce nucleic acids encoding the same or slightly different amino acid sequences of a given nucleic acid:

TABLE 3

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In other embodiments, hybridization may be achieved under conditions of, for example, 50 mM Tris-HCl (pH 8.3), 75 mM KCl, 3 mM MgC2, 10 mM dithiothreitol, at temperatures between approximately 20° C. to about 37° C. Other hybridization conditions utilized could include approximately 10 mM Tris-HCl (pH 8.3), 50 mM KCl, 1.5 1.1.M MgC12, at temperatures ranging from approximately 40° C. to about 72° C.

In certain embodiments, it will be advantageous to employ nucleic acid sequences encoding the UC proteins of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known which can be employed to provide a detection means visible to the human eye or spectrophotometrically, to identify specific hybridization with complementary nucleic acid-containing samples.

In general, it is envisioned that the hybridization probes described herein will be useful both as reagents in solution hybridization, as in PCR, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The selected conditions will depend on the particular circumstances based on the particular criteria required (depending, for example, on the G+C content, type of target nucleic acid, source of nucleic acid, size of hybridization probe, etc.). Following washing of the hybridized surface to remove non-specifically bound probe molecules, hybridization is detected, or even quantified, by means of the label.

A partial sequence may be used to identify a structurally-related gene or the full length genomic or cDNA clone from which it is derived. Those of skill in the art are well aware of the methods for generating cDNA and genomic libraries which can be used as a target for the above-described probes (Sambrook et at, 1989).

For applications in which the nucleic acid segments encoding the UC proteins are incorporated into vectors, such as plasmids, cosmids or viruses, these segments may be combined with other DNA sequences, such as promoters, polyadenylation signals, restriction enzyme sites, multiple cloning sites, other coding segments, and the like, such that their overall length may vary considerably. It is contemplated that a nucleic acid fragment of almost any length may be employed, with the total length preferably being limited by the ease of preparation and use in the intended recombinant DNA protocol.

DNA segments encoding a specific gene may be introduced into recombinant host cells and employed for expressing a specific structural or regulatory protein. Alternatively, through the application of genetic engineering techniques, subportions or derivatives of selected genes may be employed. Upstream regions containing regulatory regions such as promoter regions may be isolated and subsequently employed for expression of the selected gene.

Where an expression product is to be generated, it is possible for the nucleic acid sequence to be varied while retaining the ability to encode the same product. Reference to the codon chart, provided above, will permit those of slcill in the art to design any nucleic acid encoding for the product of a given nucleic acid.

D. Antisense constructs

In the present embodiment of the invention, nucleic acids encoding UC 28 and other UC markers or a fragment thereof may be used to produce antisense constructs targeted towards these markers. The term “antisense” is intended to refer to polynucleotide molecules complementary to a portion of a nucleic acid marker of cancer as defined herein. “Complementarj” polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

The intracellular concentration of monovalent cation is approximately 160 mM (10 mM Na⁺; 150 mM K⁺). The intracellular concentration of divalent cation is approximately 20 mM (18 mM Mg⁺; 2 mM Ca⁺⁺). The intracellular protein concentration, which would serve to decrease the volume of hybridization and, therefore, increase the effective concentration of nucleic acid species, is 150 mg/ml. Constructs can be tested in vitro under conditions that mimic these in vivo conditions.

Antisense constructs may be designed to bind to the promoter and other control regions, exons, introns or even exon-intron boundaries of a gene. It is contemplated that the most effective antisense constructs for the present invention will include regions complementary to the mRNA start site, or to those sequences encoding the UC cancer markers. One can readily test such constructs simply by testing the constructs in vitro to determine whether levels of the target protein are affected. Similarly, detrimental non-specific inhibition of protein synthesis also can be measured by determining target cell viability in vitro.

As used herein, the terms “complementary” or “antisense” mean polynucleotides that are substantially complementary over their entire length and have very few base mismatches. For example, sequences of fifteen bases in length may be termed complementary when they have a complementary nucleotide at thirteen or fourteen nucleotides out of fifteen. Naturally, sequences which are “completely complementary” will be sequences which are entirely complementary throughout their entire length and have no base mismatches.

Other sequences with lower degrees of homology also are contemplated. For example, an antisense construct which has limited regions of high homology, but also contains a non-homologous region (e.g., a ribozyme) could be designed. These molecules, though having less than 50% homology, would bind to target sequences under appropriate conditions.

As stated above, although the antisense sequences may be full length cDNA copies, or large fragments thereof, they also may be shorter fragments, or “oligonucleotides,” defined herein as polynucleotides of 50 or less bases. Although shorter oligomers (8-20) are easier to make and increase in vivo accessibility, numerous other factors are involved in determining the specificity of base-pairing. For example, both binding affinity and sequence specificity of an oligonucleotide to its complementary target increase with increasing length. It is contemplated that oligonucleotides of 8, 9, 10, 11, 12, 13, 14, 15, 16, 17, 18, 19, 20, 25, 30, 35, 40, 45, 50 or 100 base pairs will be used. While all or part of the gene sequence may be employed in the context of antisense construction, statistically, any sequence of 14 bases long should occur only once in the human genome and, therefore, suffice to specify a unique target sequence.

In certain embodiments, one may wish to employ antisense constructs which include other elements, for example, those which include C-5 propyne pyrimidines. Oligonucleotides which contain C-5 propyne analogues of uridine and cytidine have been shown to bind RNA with high affinity and to be potent antisense inhibitors of gene expression (Wagner et al., 1993).

As an alternative to targeted antisense delivery, targeted ribozymes may be used. The term “ribozyme” is refers to an RNA-based enzyme capable of targeting and cleaving particular base sequences in both DNA and RNA. Ribozymes can either be targeted directly to cells, in the form of RNA oligonucleotides incorporating ribozyme sequences, or introduced into the cell as an expression vector encoding the desired ribozymal RNA. Ribozymes may be used and applied in much the same way as described for antisense polynucleotide. Ribozyme sequences also may be modified in much the same way as described for antisense polynucleotide. For example, one could incorporate non-Watson-Crick bases, or make mixed RNA/DNA oligonucleotides, or modify the phosphodiester backbone, or modify the 2′-hydroxy in the ribose sugar group of the RNA.

Alternatively, the antisense oligo- and polynucleotides according to the present invention may be provided as RNA via transcription from expression constructs that carry nucleic acids encoding the oligo- or polynucleotides. Throughout this application, the term “expression construct” is meant to include any type of genetic construct containing a nucleic acid encoding an antisense product in which part or all of the nucleic acid sequence is capable of being transcribed.

Typical expression vectors include bacterial plasmids or phage, such as any of the pUC or Bluescript™ plasmid series or, as discussed further below, viral vectors adapted for use in eukaryotic cells.

E. Expression of Proteins from cDNAs

The cDNA species specified in SEQ ID NO:1, SEQ ID NO:5, SEQ ID NO:6, SEQ ID NO:7 and SEQ ID NO:8 may be expressed as peptide or protein. The engineering of DNA segment(s) for expression in a prokaryotic or eukaryotic system may be performed by techniques generally known to those of skill in recombinant expression. It is believed that virtually any expression system may be employed in the expression of the claimed nucleic acid sequences.

Both cDNA and genomic sequences are suitable for eukaryotic expression, as the host cell will generally process the genomic transcripts to yield functional mRNA for translation into protein. In addition, it is possible to use partial sequences for generation of antibodies against discrete portions of a gene product, even when the entire sequence of that gene product remains unknown. The generation of antibodies has been discussed earlier in the specification. Computer programs are available to aid in the selection of regions which have potential immunologic significance. For example, software capable of carrying out this analysis is readily available commercially from MacVector (IBI, New Haven, Conn.). The software typically uses standard algorithms such as the Kyte/Doolittle or Hopp/Woods methods for locating hydrophilic sequences which are characteristically found on the surface of proteins and are, therefore, likely to act as antigenic determinants.

As used herein, the terms “engineered” and “recombinant” cells are intended to refer to a cell into which an exogenous DNA segment or gene, such as a cDNA or gene has been introduced through the hand of man. Therefore, engineered cells are distinguishable from naturally occurring cells which do not contain a recombinantly introduced exogenous DNA segment or gene. Recombinant cells include those having an introduced cDNA or genomic gene, and also include genes positioned adjacent to a heterologous promoter not naturally associated with the particular introduced gene.

To express a recombinant encoded protein or peptide, whether mutant or wild-type, in accordance with the present invention one would prepare an expression vector that comprises one of the UC encoding nucleic acids under the control of, or operatively linIced to, one or more promoters. To bring a coding sequence “under the control of” a promoter, one positions the 5′ end of the transcription initiation site of the transcriptional reading frame generally between about 1 and about 50 nucleotides “downstream” (i.e., 3′) of the chosen promoter. The “upstream” promoter stimulates transcription of the DNA and promotes expression of the encoded recombinant protein. This is the meaning of “recombinant expression” in this context.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the desired gene sequence, provided such control sequences are compatible with the host cell systems.

A number of viral based expression systems may be utilized as is discussed in more detail later. In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing proteins in infected hosts.

Specific initiation signals may also be required for efficient translation of the claimed isolated nucleic acid coding sequences. These signals include the ATG initiation codon and adjacent sequences. Exogenous translational control signals, including the ATG initiation codon, may additionally need to be provided. One of ordinary slcill in the art would readily be capable of determining this and providing the necessary signals. It is well known that the initiation codon must be in-frame (or in-phase) with the reading frame of the desired coding sequence to ensure translation of the entire insert. These exogenous translational control signals and initiation codons may be of a variety of origins, both natural and synthetic. The efficiency of expression may be enhanced by the inclusion of appropriate transcription enhancer elements or transcription terminators (Bittner et al., 1987).

In eukaryotic expression, one will also typically desire to incorporate into the transcriptional unit an appropriate polyadenylation site (e.g., 5′-AATAAA-3′) if one was not contained within the original cloned segment. Typically, the poly A addition site is placed about 30 to 2000 nucleotides “downstream” of the termination site of the protein at a position prior to transcription termination.

For long-term, high-yield production of recombinant proteins, stable expression is preferred. For example, cell lines that stably express constructs encoding proteins may be engineered. Rather than using expression vectors that contain viral origins of replication, host cells may be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn may be cloned and expanded into cell lines.

A number of selection systems may be used, including but not limited to, the herpes simplex virus thymidine kinase (Wigler et al., 1977), hypoxanthine-guanine phosphoribosyltransferase (Szybalska et al., 1962) and adenine phosphoribosyltransferase genes (Lowy et al., 1980), in tk-, hgprt- or aprt-cells, respectively. Also, antimetabolite resistance may be used as the basis of selection for dhfr, that confers resistance to methotrexate (Wigler et al., 1980; O'Hare et al., 1981); gpt, that confers resistance to mycophenolic acid (Mulligan et al., 1981); neo, that confers resistance to the aminoglycoside G-418 (Colberre-Garapin et al., 1981); and hygro, that confers resistance to hygromycin (Santerre et al., 1984).

In the present embodiment of the invention, the UC proteins encoding nucleic acids of the present invention may be “overexpressed”, i.e., expressed in increased levels relative to its natural expression in human cells, or even relative to the expression of other proteins in the recombinant host cell. Such overexpression may be assessed by a variety of methods, including radio-labeling and/or protein purification. However, simple and direct methods are preferred, for example, those involving SDS/PAGE and protein staining or Western blotting, followed by quantitative analyses, such as densitometric scanning of the resultant gel or blot. A specific increase in the level of the recombinant protein or peptide in comparison to the level in natural human cells is indicative of overexpression, as is a relative abundance of the specific protein in relation to the other proteins produced by the host cell and, e.g., visible on a gel.

F. Viral Vectors as Delivery Vehicles

1. Adenoviral Vectors

Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a recombinant gene construct that has been cloned therein.

The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promotdi possess a 5′-tripartite leader (TPL) sequence which makes them preferred mRNA's for translation.

Recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

In nature, adenovirus can package approximately 105% of the wild-type genome (Ghosh-Choudhury et al., 1987), providing capacity for about 2 extra kb of DNA. Helper cell lines derived from human cells such as human embryonic kidney cells, muscle cells, hematopoietic cells or other human embryonic mesenchymal or epithelial cells may be used to make the construct. Alternatively, the helper cells may be derived from the cells of other mammalian species that are permissive for human adenovirus. Such cells include, e.g., Vero cells or other monkey embryonic mesenchymal or epithelial cells.

The adenovirus vector may be replication defective, or at least conditionally defective, the nature of the adenovirus vector is not believed to be crucial to the successful practice of the invention. The adenovirus may be of any of the 42 different known serotypes or subgroups A-F.

Adenovirus growth and manipulation is known to those of skill in the art, and exhibits broad host range in vitro and in vivo. This group of viruses can be obtained in high titers, e.g., 109-1011 plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

2. Retroviral Vectors

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provinis and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, packaging cell lines are available that should greatly decrease the likelihood of recombination (Markowitz et al., 1988; Hersdorffer et al., 1990).

3. AAV Vectors

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin, et al., 1984; Laughlin, et al., 1986; Lebkowski, et al., 1988; McLaughlin, et al., 1988), which means it is applicable for use with the present invention. Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Lebkowski et al., 1988; Samulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al., 1995).

4. Other Viral Vectors

Other viral vectors may be employed as constructs in the present invention. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990).

A molecularly cloned strain of Venezuelan equine encephalitis (VEE) virus has been genetically refined as a replication competent vaccine vector for the expression of heterologous viral proteins. Studies have demonstrated that VEE infection stimulates potent CTL responses and has been suggested that VEE may be an extremely useful vector for immunizations (Caley et al., 1997). It is contemplated in the present invention, that VEE virus may be useful in targeting dendritic cells.

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In still further embodiments of the present invention, the nucleic acid encoding human UC28 or other UC markers mentioned herein is housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. Such modifications permit specific infection of cancer and/or hyperproliferative cells via specific receptors present on these cells.

For example, targeting of recombinant retroviruses was designed in which biotinylated antibodies against a retroviral envelope protein and against a specific cell receptor were used. The antibodies were coupled via the biotin components by using streptavidin (Roux et al., 1989).

Using antibodies against major histocompatibility complex class I and class II antigens, they demonstrated the infection of a variety of human cells that bore those surface antigens with an ecotropic virus in vitro (Roux et al., 1989).

G. Other Delivery Vehicles

In certain broad embodiments of the invention, the antisense oligo- or polynucleotides and/or expression vectors may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are cationic lipid-nucleic acid complexes, such as lipofectamine-nucleic acid complexes.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression vectors have been successfully employed in transfer and expression of a polynucleotide in vitro and in vivo, then they are applicable for the present invention. Where a bacterial promoter is employed in the DNA construct, it also will be desirable to include within the liposome an appropriate bacterial polymerase.

“Liposome” is a generic term encompassing a variety of single and multilamellar lipid vehicles formed by the generation of enclosed lipid bilayers. Phospholipids are used for preparing the liposomes according to the present invention and can carry a net positive charge, a net negative charge or are neutral. Dicetyl phosphate can be employed to confer a negative charge on the liposomes, and stearylamine can be used to confer a positive charge on the liposomes.

Lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform, chloroform/methanol or t-butanol can be stored at about −20° C. Preferably, chloroform is used as the only solvent since it is more readily evaporated than methanol.

Phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition, because of the instability and leakiness of the resulting liposomes.

Liposomes used according to the present invention can be made by different methods. The size of the liposomes varies depending on the method of synthesis. A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules will form a bilayer, known as a lamella, of the arrangement XY-YX.

Liposomes within the scope of the present invention can be prepared in accordance with known laboratory techniques. In one preferred embodiment, liposomes are prepared by mixing liposomal lipids, in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In the alternative, liposomes can be prepared in accordance with other known laboratory procedures: the method of Bangham et al. (1965), the contents of which are incorporated herein by reference; the method of Gregoriadis, as described in DRUG CARRIERS IN BIOLOGY AND MEDICINE, G. Gregoriadis ed. (1979) pp. 287-341, the contents of which are incorporated herein by reference; the method of Deamer and Uster (1983), the contents of which are incorporated by reference; and the reverse-phase evaporation method as described by Szoka and Papahadjopoulos (1978). The aforementioned methods differ in their respective abilities to entrap aqueous material and their respective aqueous space-to-lipid ratios.

The dried lipids or lyophilized liposomes prepared as described above may be reconstituted in a solution of nucleic acid and diluted to an appropriate concentration with an suitable solvent, e.g., DPBS. The mixture is then vigorously shaken in a vortex mixer. Unencapsulated nucleic acid is removed by centrifugation at 29,000×g and the liposomal pellets washed. The washed liposomes are resuspended at an appropriate total phospholipid concentration, e.g., about 50-200 mM. The amount of nucleic acid encapsulated can be determined in accordance with standard methods. After determination of the amount of nucleic acid encapsulated in the liposome preparation, the liposomes may be diluted to appropriate concentration and stored at 4° C. until use.

In a preferred embodiment, the lipid dioleoylphosphatidylcholine is employed. Nuclease-resistant oligonucleotides were mixed with lipids in the presence of excess t-butanol. The mixture was vortexed before being frozen in an acetone/dry ice bath. The frozen mixture was lyophilized and hydrated with Hepes-buffered saline (1 mM Hepes, 10 mM NaCl, pH 7.5) overnight, and then the liposomes were sonicated in a bath type sonicator for 10 to 15 min. The size of the liposomal-oligonucleotides typically ranged between 200-300 nm in diameter as determined by the submicron particle sizer autodilute model 370 (Nicomp, Santa Barbara, Calif.).

In a further embodiment of the invention, the gene construct may be entrapped in a liposome or lipid formulation. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Lipid-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of lipid-mediated delivery and expression of foreign DNA in cultured chick embryo, HeLa and hepatoma cells.

Lipid based non-viral formulations provide an alternative to adenoviral gene therapies. Although many cell culture studies have documented lipid based non-viral gene transfer, systemic gene delivery via lipid based formulations has been limited. A major limitation of non-viral lipid based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in lipid vehicle stability in the presence and absence of serum proteins. The interaction between lipid vehicles and serum proteins has a dramatic impact on the stability characteristics of lipid vehicles (Yang and Huang, 1997). Cationic lipids attract and bind negatively charged serum proteins. Lipid vehicles associated with serum proteins are either dissolved or taken up by macrophages leading to their removal from circulation. Current in vivo lipid delivery methods use subcutaneous, intradermal, intratumoral, or intracranial injection to avoid the toxicity and stability problems associated with cationic lipids in the circulation. The interaction of lipid vehicles and plasma proteins is responsible for the disparity between the efficiency of in vitro (Feigner et al., 1987) and in vivo gene transfer (Zhu et al., 1993; Solodin et al., 1995; Thierry et al., 1995; Aksentijevich et al, 1996).

‘The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Lipid encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating cancers.

### IV. Methods of Inhibiting Cancer Cells

A. Differentiation/Inhibition Therapy

The present invention concerns a therapy that malces use of a differentiation inducing agent in combination with an inhibitory agent for the treatment of cancer.

Cell differentiation is the process by which a daughter cell is different from its parent either through its cytoplasmic or its nuclear information. ‘The changes are often expressed through turning genes on, and off and may be irreversible. An agent that induces the differentiation in cells is defined as a cell differentiation inducing agent.

UC28 is differentially expressed in cancer cells with significant up regulation noted in cancer tissues over normal and benign disease states. Additionally, the UC28 protein is expressed on the cell membrane and its expression is significantly increased in malignant cancer cells exposed to treatment with differentiating agents such as sodium phenylbutryate (SPB) but not in normal or non-cancer cells.

The therapy contemplates the use of an effective amount of differentiation inducing agent that preferentially induces expression of UC 28 membrane protein in cancerous cells. ‘The present invention further contemplates the use of such an agent with an effective amount of an inhibitory agent that may inhibit the expression of UC28 protein as encoded by SEQ ID NO:1 and other UC proteins as encoded by SEQ ID NO:5, SEQ ID NO:6, SEQ ID NO:7 and SEQ ID NO:8.

A therapy that uses UC 28 expressing cells as a target for cancer agents, such as toxins is also an embodiment of the invention.

1. Differentiation Agents

The present invention contemplates the use of Sodium Phenylbuyrate (SPB) which is both a differentiation inducing agent in malignant cells and a growth inhibitor. Other differentiation inducing agents that are contemplated for use by this invention are SAHA, sodium phenylacetate, 13 cis-Retinoic acid (CRA), and other retinoids short chain fatty acids, DMSO, N-Methylformamide, Vitamine D3, Vitamine D3 analogs like maxacalcitol also known as 22-oxacalcitriol, Vitamine E, Estrogens, glucocorticoids, Protein kinase C(PKC) activators, PKC inhibitors, thiazolidinedione, troglitazones, oxacalcitriol or onconase, retinoids, Interferons, Tumor Necrosis Factors.

These may be used in combination with an inhibitory agent, which may be an immunotoxin, an anti-UC28 antibody, an antisense construct or a ribozyme against UC 28 protein that may function as an inhibitor of UC28 protein. The inhibitor may also form a part of a fusion or chimeric protein. The invention further contemplates the use of such a therapy with other UC markers as encoded by SEQ ID NO:5, SEQ ID NO:6, SEQ ID NO:7, SEQ ID NO:8.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired.

Precise amounts of the therapeutic composition also depend on the judgment of the practitioner and are peculiar to each individual. Factors affecting dose include physical and clinical state of the patient, the route of administration and the potency, stability and toxicity of the particular therapeutic substance. For the instant application, it is envisioned that the amount of differentiation agent that forms a part of a therapeutic composition comprising a unit dose will range from about 2-100 mM.

Further, it is also contemplated that the administration of a combination of any therapeutic agent for the treatment of cancer with a differentiation/inhibitor therapy may enhance the efficacy of the treatment.

Some of the differentiation agents are descibed below:

### Sodium Phenylbutyrate (SPB)

Glutamine is a non-essential amino acid and the major nitrogen source for nucleic acid and protein synthesis. It is also an important energy substrate in rapidly dividing cells. Tumor cells are significantly more sensitive to glutamine depletion than normal cells, as they function on limiting levels of glutamine availability due to their increased utilization and accelerated catabolism. The glutamine depleting enzyme glutaminase, as well as some glutamine antimetabolites have shown promising antineoplastic activity, but their clinical usefulness has been limited by their unacceptable side effects and toxicity.

Phenylbutyrate depletes the cells of glutamine without affecting the glutamine utilizing enzymes. In its metabolized form it is capable of conjugating glutamine to yield PAG (phenylacetyl glutamine), which is then excreted in the urine, and the tumor cells will not have enough “fuel” to continue to grow and multiply. Normal cells are not affected by the used dosages. It has been shown (Samid 1992) that Phenylbutyrate arrests tumor growth and induces differentiation of pre-malignant and malignant cells through this non-toxic mechanism. Phenylbutyrate has been shown to be a non-toxic differentiation inducer, promoting maturation of various types of malignant cells. Maturation makes the cells less aggressive, causing them to cease dividing and eventually die (Carducci et al., 1996; Carducci et al., 1997; Melchoir et al., 1999; Candido et al., 1978; Lea et al., 1998; Gorospe et al., 1996; Richon et al., 1998; Sambucetti et al., 1999).

### Onconase

Onconase is a cytotoxic ribonuclease derived from the eggs (oocytes) and embryonic stem cells of the leopard frog Rana pipiens inhibits cancer cell growth and viral replication. This protein is active against a wide variety of tumor cell types (e.g. breast, kidney, lung, prostate), it is especially active against carcinomas (i.e. solid tumors cancers) which may account for about 90% of all cancers. Also clinical trial data have established onconase exhibits low toxicity in humans (Halicka et al., 1996).

### Troglitazone

Troglitazone is known to help diabetics indirectly by binding to a protein called Peroxisome Proliferator Activated Receptor-gamma (PPAR-gamma) that, among other things, helps speed the maturation of fat cells, making them more effective at removing glucose from the blood. The drug's ability to age cells makes it possible to use it to treat cancer, in which cells gain a kind of immortality and reproduce uncontrollably.

### SAHA

Hybrid Polar Cytodifferentiation (HPC) agents represent a novel class of anticancer compounds which act by inducing terminal differentiation and/or apoptosis. Suberanilohydroxamic acid (SAHA) belongs to this class (Cohen et al., 1999, Butler et al., 2000, Huang and Pardee, 2000). SAHA is an inhibitor of histone deacetylases (HDACs) which are involved in cell-cycle progression and differentiation and their deregulation in several cancers (Finnin et al., 1999, Butler et al., 2000, Huang et al., 2000). The critical site on SAHA is the hydroxyaminic moiety.

B. Induction of Immune Response

The section on antibody generation discussed the monoclonal and polyclonal antibodies. In the present invention these antibodies may be used to induce an immune response.

1. Vaccines

The present invention includes methods for preventing the development of cancer in both infected and uninfected persons who express one or more UC markers. As such, the invention contemplates vaccines for use in both active and passive immunization embodiments. Immunogenic compositions, proposed to be suitable for use as a vaccine, may be prepared most readily directly from immunogenic UC marker peptides or proteins. All or part of any UC marker is contemplated for use as a vaccine. Furthemore, more than one UC marker may be employed. Any of the methods or compositions discussed with respect to proteinaceous compositions may be applied with respect to vaccines. Vaccines may be produced recombinantly or they may be synthetically produced. Preferably the antigenic material is extensively dialyzed to remove undesired small molecular weight molecules and/or lyophilized for more ready formulation into a desired vehicle. The methods described and claimed in U.S. Pat. No. 6,210,662 are contemplated as part of the invention; this reference is specifically incorporated by reference. Furthermore, it is contemplated that antigen presenting cells, such as dendritic cells, may be employed as part of a vaccine, as described in U.S. Pat. No. 6,121,044, which is specifically incorporated by reference.

Alternatively, other viable and important options for a peptide-based vaccine involve introducing the peptide sequences as nucleic acids, either as direct DNA vaccines or recombinant vaccinia virus-based polyepitope vaccine. The use of nucleic acid sequences as vaccines is described in U.S. Pat. Nos. 5,958,895 and 5,620,896, which are incorporated by reference.

Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, or adjuvants that enhance the effectiveness of the vaccines.

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

The UC marker-derived peptides and UC marker-encoded DNA constructs of the present invention may be formulated into the vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be therapeutically effective and immunogenic. The quantity to be administered depends on the subject to be treated, including, e.g., the capacity of the individual's immune system to synthesize antibodies and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as about 0.05 to about 0.1% solution in phosphate buffered saline, admixture with synthetic polymers of sugars (Carbopol0) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin, mixture with bacterial cells such as C. parvum or endotoxins or lipopolysaccharide components of Gram-negative bacteria, emulsion in physiologically acceptable oil vehicles such as mannide mono-oleate (Aracel A), or emulsion with a 20% solution of a perfluorocarbon (Fluosol-DA®) used as a block substitute may also be employed.

In many instances, it will be desirable to have multiple administrations of the vaccine, usually not exceeding six vaccinations, more usually not exceeding four vaccinations and preferably one or more, usually at least about three vaccinations. The vaccinations will normally be at from two to twelve week intervals, more usually from three to five week intervals. Periodic boosters at intervals of 1-5 years, usually three years, will be desirable to maintain protective levels of the antibodies. The course of the immtmization may be followed by assays for antibodies for the supernatant antigens. The assays may be performed by labeling with conventional labels, such as radionuclides, enzymes, fluorescents, and the like. These teclmiques are well known and may be found in a wide variety of patents, such as U.S. Pat. Nos. 3,791,932; 4,174,384 and 3,949,064, as illustrative of these types of assays.

C. Combination Cancer Therapy

A wide variety of cancer therapies, known to one of slcill in the art, may be used in combination with the differentiation/inhibition therapy contemplated in the present invention towards UC markers. Further, the use of this combination is also contemplated for targeting other UC markers. Thus, in order to increase the effectiveness of the anticancer therapy using a polypeptide, or expression construct coding therefor, it may be desirable to combine these compositions with other agents effective in the treatment of cancer such as but not limited to those described below. Such a therapy directly involving a UC marker will be termed as “UC based therapy” throughout the application.

For example, one can use the UC based therapy as in differentiation/inhibition therapy in conjunction with surgery and/or chemotherapy, and/or immunotherapy, and/or other gene therapy, and/or radiotherapy, and/or local heat therapy. Thus, one can use one or several of the standard cancer therapies existing in the art in addition with the UC-based therapies of the present invention. All other non-UC-based cancer therapies are referred to herein as “other cancer therapies”.

The other cancer therapy may precede or follow the UC-based therapy by intervals ranging from minutes to days to weeks. In embodiments where the other cancer therapy and the UC-based therapy are administered together, one would generally ensure that a significant period of time did not expire between the time of each delivery. In such instances, it is contemplated that one would administer to a patient both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the other cancer therapy and the UC-based therapy will be required to achieve complete cancer cure. Various combinations may be employed, where the other cancer therapy is “A” and the UC28-based therapy treatment is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A AB/BB B/A/B/B B/B/A/B

Other combinations also are contemplated.

In addition, the UC-based therapy can be administered to a patient in conjunction with other therapeutic methods. The exact dosages and regimens can be suitable altered by those of ordinary skill in the art.

1. Radiotherapeutic Agents

Radiotherapeutic agents and factors include radiation and waves that induce DNA damage for example, y-irradiation, X-rays, UV-irradiation, microwaves, electronic emissions, radioisotopes, and the like. Therapy may be achieved by irradiating the localized tumor site with the above described forms of radiations.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

2. Surgery

Approximately 60% of persons with cancer will undergo surgery of some type, which includes preventative, diagnostic or staging, curative and palliative surgery. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy, such as with proteinaceous compositions encoding targeting agents against UC markers. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

3. Chemotherapeutic Agents

Agents that affect DNA function are defined as chemotherapeutic agents, for example, agents that directly cross-link DNA, agents that intercalate into DNA, and agents that lead to chromosomal and mitotic aberrations by affecting nucleic acid synthesis. Some examples of chemotherapeutic agents include antibiotic chemotherapeutics such as, Doxorubicin, Daunorubicin, Mitomycin (also known as mutamycin and/or mitomycin-C), Actinomycin D (Dactinomycin), Bleomycin, Plicomycin. Plant alkaloids such as Taxol, Vincristine, Vinblastine. Miscellaneous agents such as Cisplatin, VP16, Tumor Necrosis Factor. Alkylating Agents such as, Carmustine, Melphalan (also known as alkeran, L-phenylalanine mustard, phenylalanine mustard, L-PAM, or L-sarcolysin, is a phenylalanine derivative of nitrogen mustard), Cyclophosphamide, Chlorambucil, Busulfan (also known as myleran), Lomustine. And other agents for example, Cisplatin (CDDP), Carboplatin, Procarbazine, Mechlorethamine, Camptothecin, Ifosfamide, Nitrosurea, Etoposide (VP16), Tamoxifen, Raloxifene, Estrogen Receptor Binding Agents, Gemcitabien, Navelbine, Farnesyl-protein transferase inhibitors, Transplatinum, 5-Fluorouracil, and Methotrexate, Temazolomide (an aqueous form of DTIC), or any analog or derivative variant of the foregoing.

4. Immunotherapy

Immunotherapeutics, generally, rely on the use of immune effector cells and molecules to target and destroy cancer cells. The immune effector may be, for example, an antibody specific for some marker on the surface of a tumor cell. In the present invention, the tumor marker is a UC marker. Other common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. UC marker encoding gene transfer to cancer cells causes cell death and apoptosis. The apoptotic cancer cells are scavenged by reticuloendothelial cells including dendritic cells and macrophages and presented to the immune system to generate antitumor immunity (Rovere et al., 1999; Steinman et al., 1999). Immune stimulating molecules may be provided as immune therapy: for example, cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with proteinaceous compositions that act as targeting agents against UC markers will enhance anti-tumor effects. Thus one may use (i) Passive Immunotherapy which includes: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow; and/or (ii) Active Immunotherapy wherein an antigenic peptide, polypeptide or protein, or an autologous or allogenic tumor cell composition or “vaccine” is administered, generally with a distinct bacterial adjuvant (Ravindranath & Morton, 1991) and/or (iii) Adoptive Immunotherapy wherein the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

In another aspect of the invention, passive cellular dendritic cells immunotherapy may be combined with differentiation therapy using UC28 and other peptides mentioned earlier in the application. In this approach, a UC28 peptide may be used to sensitize patient's dendritic cells in vitro. Following this, the patient who is already being treated with differentiation therapy may be administered the sensitized dendritic cells. The combined effect would take advantage of antigen sensitization and in vivo upregulation of the UC28 protein making this approach to targeted immunotherapy even more effective (Dendreon Corp., Seattle).

5. Gene Therapy

In yet another embodiment, the other treatment is a secondary gene therapy in which a second therapeutic polynucleotide is administered before, after, or at the same time as the first therapeutic polynucleotide encoding a proteinaceous composition used as a targeting agent against a UC marker. Delivery of a vector encoding a UC polypeptide in conjunction with a second vector encoding one of the following gene products will have a combined anti-hyperproliferative effect on target tissues. Alternatively, a single vector encoding both genes may be used. A variety of proteins are encompassed within the invention, some of which are described elsewhere in the specification under the sections: Inducers of cellular proliferation, inhibitors of cellular proliferation, regulators of programmed cell death, and other agents. Table 4 lists various genes that may be targeted for gene therapy of some form in combination with the present invention.

TABLE 4
Oncogenes

| | | | |
|---|---|---|---|
| Gene | Source | Human Disease | Function |

Growth Factors

| | | |
|---|---|---|
| HST/KS | Transfection | FGF family member |
| INT-2 | MMTV promoter | FGF family member |
| | Insertion | |
| INTI/WNTI | MMTV promoter | Factor-like |
| | Insertion | |
| SIS | Simian sarcoma virus | PDGF B |

Receptor Tyrosine Kinases

| | | | |
|---|---|---|---|
| ERBB/HER | Avian erythroblastosis | Amplified, deleted | EGF/TGF-/ |
| | virus; ALV promoter | squamous cell cancer; | Amphiregulin/ |
| | insertion; amplified | glioblastoma | etacellulin receptor |
| | human tumors | | |
| ERBB-2/NEU/HER-2 | Transfected from rat | Amplified breast, | Regulated by NDF/ |
| | Glioblastomas | ovarian, gastric | Heregulin and EGF- |
| | | cancers | Related factors |
| FMS | SM feline sarcoma virus | | CSF-1 receptor |
| KIT | HZ feline sarcoma virus | | MGF/Steel receptor |
| | | | Hematopoieis |
| TRK | Transfection from | | NGF (nerve growth |
| | human colon cancer | | Factor) receptor |
| MET | Transfection from | | Scatter factor/HGF |
| | human osteosarcoma | | Receptor |
| RET | Translocations and | Sporadic thyroid cancer; | Orphan receptor Tyr |
| | point mutations | familial medullary | Kinase |
| | | thyroid cancer; | |
| | | multiple endocrine | |
| | | neoplasias 2A and 2B | |
| ROS | URII avian sarcoma | | Orphan receptor Tyr |
| | Virus | | Kinase |
| PDGF receptor | Translocation | Chronic | TEL(ETS-like |
| | | Myelomonocytic | transcription factor)/ |
| | | Leukemia | PDGF receptor gene |
| | | | Fusion |
| TGF- receptor | | Colon carcinoma | |
| | | mismatch mutation | |
| | | target | |

NONRECEPTOR TYROSINE KINASES

| | | | |
|---|---|---|---|
| ABI | Abelson Mul.V | Chronic myelogenous | Interact with RB, RNA |
| | | leukemia translocation | polymerase, CRK, |
| | | with BCR | CBL |
| FPS/FES | Avian Fujinami SV; GA | | |
| | FeSV | | |
| LCK | Mul.V (murine | | Src family; T-cell |
| | leukemia virus) | | signaling; interacts |
| | promoter insertion | | CD4/CD8 T-cells |
| SRC | Avian Rous sarcoma | | Membrane-associated |
| | Virus | | Tyr kinase with |
| | | | signaling function; |
| | | | activated by receptor |
| | | | kinases |
| YES | Avian Y73 virus | | Src family; signaling |

SER/THRPROTEIN KINASES

| | | |
|---|---|---|
| AKT | AKT8 murine | Regulated by PI(3)K?; |
| | retrovirus | regulate 70-kd S6 k? |
| MOS | Maloney murine SV | GVBD; cystostatic |
| | | factor; MAP kinase |
| | | kinase |
| PIM-1 | Promoter insertion | |
| | Mouse | |
| RAF/MIL | 3611 murine SV; MH2 | Signaling in RAS |
| | avian SV | Pathway |

MISCELLANEOUS CELL SURFACE¹

| | | | |
|---|---|---|---|
| APC | Tumor suppressor | Colon cancer | Interacts with catenins |
| DCC | Tumor suppressor | Colon cancer | CAM domains |
| E-cadherin | Candidate tumor | Breast cancer | Extracellular homotypic |
| | Suppressor | | binding; intracellular |
| | | | interacts with catenins |
| PTC/NBCCS | Tumor suppressor and | Nevoid basal cell cancer | 12 transmembrane |
| | Drosophilia | syndrome (Gorline | domain; signals |
| | homology | syndrome) | through Gli homogue |
| | | | CI to antagonize |
| | | | hedgehog pathway |
| TAN-1 Notch homologue | Translocation | T-ALI. | Signaling? |

MISCELLANEOUS SIGNALING

| | | | |
|---|---|---|---|
| BCL-2 | Translocation | B-cell lymphoma | Apoptosis |
| CBL | Mu Cas NS-1 V | | Tyrosine- |
| | | | Phosphorylated RING |
| | | | finger interact Abl |
| CRK | CT1010 ASV | | Adapted SH2/SH3 |
| | | | interact Abl |
| DPC4 | Tumor suppressor | Pancreatic cancer | TGF--related signaling |
| | | | Pathway |
| MA S | Transfection and | | Possible angiotensin |
| | Tumorigenicity | | Receptor |
| NCK | | | Adaptor SH2/SH3 |

GUANINE NUCLEOTIDE EXCHANGERS AND BINDING PROTEINS

| | | | |
|---|---|---|---|
| BCR | | Translocated with ABL | Exchanger; protein |
| | | in CML | Kinase |
| DBL | Transfection | | Exchanger |
| GSP | | | |
| NF-1 | Hereditary tumor | Tumor suppressor | RAS GAP |
| | Suppressor | Neurofibromatosis | |
| OST | Transfection | | Exchanger |
| Harvey-Kirsten, N-RAS | HaRat SV; Ki RaSV; | Point mutations in many | Signal cascade |
| | Balb-MoMuSV; | human tumors | |
| | Transfection | | |
| VAV | Transfection | | S112/S113; exchanger |

NUCLEAR PROTEINS AND TRANSCRIPTION FACTORS

| | | | |
|---|---|---|---|
| BRCA1 | Heritable suppressor | Mammary cancer/ | Localization unsettled |
| | | ovarian cancer | |
| BRCA2 | Heritable suppressor | Mammary cancer | Function unknown |
| ERBA | Avian erythroblastosis | | thyroid hormone |
| | Virus | | receptor (transcription) |
| ETS | Avian E26 virus | | DNA binding |
| EVII | MuLV promotor | AML | Transcription factor |
| | Insertion | | |
| FOS | FBI/FBR murine | | 1 transcription factor |
| | osteosarcoma viruses | | with c-JUN |
| GLI | Amplified glioma | Glioma | Zinc finger; cubitus |
| | | | interruptus homologue |
| | | | is in hedgehog |
| | | | signaling pathway; |
| | | | inhibitory link PTC |
| | | | and hedgehog |
| HMGI/LIM | Translocation t(3:12) | Lipoma | Gene fusions high |
| | t(12:15) | | mobility group HMGI- |
| | | | C (XT-hook) and |
| | | | transcription factor |
| | | | LIM or acidic domain |
| JUN | ASV-17 | | Transcription factor |
| | | | AP-1 with FOS |
| MLIJVHRX + EL1/MEN | Translocation/fusion | Acute myeloid | Gene fusion of DNA- |
| | ELL with MLL | leukemia | binding and methyl |
| | Trithorax-like gene | | transferase MLL with |
| | | | ELI RNA pol II |
| | | | elongation factor |
| MYB | Avian myeloblastosis | | DNA binding |
| | Virus | | |
| MYC | Avian MC29; | Burkitt's lymphoma | DNA binding with |
| | Translocation B-cell | | MAX partner; cyclin |
| | Lymphomas; promoter | | regulation; interact |
| | Insertion avian leukosis | | RB?; regulate |
| | Virus | | apoptosis? |
| N-MYC L-MYC | Amplified | Neuroblastoma | |
| | | Lung cancer | |
| REL | Avian | | NF-B family |
| | Retriculoendotheliosis | | transcription factor |
| | Virus | | |
| SKI | Avian SKV770 | | Transcription factor |
| | Retrovirus | | |
| VHL | Heritable suppressor | Von Hippel-Landau | Negative regulator or |
| | | syndrome | elongin; transcriptional |
| | | | elongation complex |
| WT-1 | | Wilm's tumor | Transcription factor |

CELL CYCLE/DNA DAMAGE RESPONSE

| | | | |
|---|---|---|---|
| ATM | Hereditary disorder | Ataxia-telangiectasia | Protein/lipid kinase |
| | | | homology; DNA |
| | | | damage response |
| | | | upstream in P53 |
| | | | pathway |
| BCL-2 | Translocation | Follicular lymphoma | Apoptosis |
| | | Fanconi's anemia | |
| FACC | Point mutation | group | |
| | | C (predisposition | |
| | | Leukemia | |
| MDA-7 | Fragile site 3p14.2 | Lung carcinoma | Histidine triad-related |
| | | | diadenosine 5-,3- t e |
| | | | traphosphate |
| | | | asymmetric hydrolase |
| hMLI/MutL | | HNPCC | Mismatch repair; MutL |
| | | | Homologue |
| hMSH2/MutS | | HNPCC | Mismatch repair; MutS |
| | | | Homologue |
| hPMS1 | | HNPCC | Mismatch repair; MutL |
| | | | Homologue |
| hPMS2 | | HNPCC | Mismatch repair; MutL |
| | | | Homologue |
| INK4/MTS1 | Adjacent INK-4B at | Candidate MTS I | p16 CDK inhibitor |
| | 9p21; CDK complexes | Suppressor and | |
| | | MLM melanoma | |
| | | gene | |
| INK4B/MTS2 | | Candidate suppressor | p15 CDK inhibitor |
| MDM-2 | Amplified | Sarcoma | Negative regulator p53 |
| p53 | Association with SV40 | Mutated >50% human | Transcription factor; |
| | T antigen | tumors, including | checkpoint control; |
| | | hereditary Li- | apoptosis |
| | | Fraumeni syndrome | |
| PRAD1/BCL1 | Translocation with | Parathyroid adenoma; | Cyclin D |
| | Parathyroid hormone | B-CLL | |
| | or IgG | | |
| RB | Hereditary | Retinoblastoma; | Interact cyclin/cdk; |
| | Retinoblastoma; | Osteosarcoma; breast | regulate E2F |
| | Association with many | cancer; other | transcription factor |
| | DNA virus tumor | sporadic cancers | |
| | Antigens | | |
| XPA | | Xeroderma | Excision repair; photo- |
| | | pigmentosum; skin | product recognition; |
| | | cancer predisposition | zinc finger |

One of the therapeutic embodiments contemplated by the present inventors is the intervention, at the molecular level, in the events involved in the tumorigenesis of some cancers. Specifically, the present inventors intend to provide, to a cancer cell, an expression construct capable of providing a polypeptide that can target UC28 or other UC peptides mentioned herein to that cell.

Those of skill in the art are well aware of how to apply gene delivery to in vivo and ex vivo situations. For viral vectors, one generally will prepare a viral vector stock. Depending on the kind of virus and the titer attainable, one will deliver 1 to 100, 10 to 50, 100-1000, or up to 1×10⁴, 1×10⁵, 1×10⁶, 1×10⁷, 1×10⁸, 1×10⁹, 1×10¹⁰, 1×10¹¹, or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for various tumor types. The section below on routes contains an extensive list of possible routes. For practically any tumor, systemic delivery is contemplated. This will prove especially important for attacking microscopic or metastatic cancer. Where discrete tumor mass, or solid tumor, may be identified, a variety of direct, local and regional approaches may be taken. For example, the tumor may be directly injected with the expression vector. A tumor bed may be treated prior to, during or after resection. Following resection, one generally will deliver the vector by a catheter left in place following surgery. One may utilize the tumor vasculature to introduce the vector into the tumor by injecting a supporting vein or artery. A more distal blood supply route also may be utilized.

The method of treating cancer includes treatment of a tumor as well as treatment of the region near or around the tumor. In this application, the term “residual tumor site” indicates an area that is adjacent to a tumor. This area may include body cavities in which the tumor lies, as well as cells and tissue that are next to the tumor.

In a different embodiment, ex vivo gene therapy is contemplated. This approach is particularly suited, although not limited, to treatment of bone marrow associated cancers. In an ex vivo embodiment, cells from the patient are removed and maintained outside the body for at least some period of time. During this period, a therapy is delivered, after which the cells are reintroduced into the patient; hopefully, any tumor cells in the sample have been killed.

### V. PHARMACEUTICAL COMPOSITIONS AND ROUTES OF ADMINISTRATION

The present invention contemplates a method of preventing the development of cancer. In some embodiments, pharmaceutical compositions are administered to a subject. Different aspects of the present invention involve administering an effective amount of an aqueous compositions. In another embodiment of the present invention, UC marker polypeptides or peptides may be administered to the patient to prevent the development of cancer. Alternatively, an expression vector encoding such polypeptides or peptides may be given to a patient as a preventative treatment. Additionally, such compounds can be administered in combination with differentiation agents. Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal, or human, as appropriate. As use,d herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

In addition to the compounds formulated for parenteral administration, such as those for intravenous or intramuscular injection, other pharmaceutically acceptable forms include, e.g., tablets or other solids for oral administration; time release capsules; and any other form currently used, including cremes, lotions, mouthwashes, inhalants and the like.

The active compounds of the present invention can be formulated for parenteral administration, e.g., formulated for injection via the intravenous, intramuscular, sub-cutaneous, or even intraperitoneal routes. The preparation of an aqueous composition that contains a compound or compounds that increase the expression of UC marker protein will be known to those of skill in the art in light of the present disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

In certain cases, the therapeutic formulations of the invention also may be prepared in forms suitable for topical administration, such as in cremes and lotions. These forms may be used for treating skin-associated diseases, such as various sarcomas.

Administration of therapeutic compositions according to the present invention will be via any common route so long as the target tissue is available via that route. This includes oral, nasal, buccal, rectal, vaginal, mucosal, or topical. Alternatively, administration will be by orthotopic, intradermal subcutaneous, intramuscular, intraperitoneal, intravaginal, intranasal, or intravenous injection. Such compositions would normally be administered as pharmaceutically acceptable compositions that include physiologically acceptable carriers, buffers or other excipients. For treatment of conditions of the lungs, aerosol delivery to the lung is contemplated. Volume of the aerosol is between about 0.01 ml and 0.5 ml. Similarly, a preferred method for treatment of colon-associated disease would be via enema. Volume of the enema is between about 1 ml and 100 ml.

In certain embodiments, it may be desirable to provide a continuous supply of therapeutic compositions for a period of time to the patient. The time frame includes administration for one or more hours, one or more days, one or more weeks, or one or more months, with a possible hiatus during that time period. For intravenous or intraarterial routes, this is accomplished by drip system. For topical applications, repeated application would be employed. For various approaches, delayed release formulations could be used that provided limited but constant amounts of the therapeutic agent over and extended period of time. For internal application, continuous perfusion, for example with a synthetic UC peptide or a fragment thereof, of the region of interest may be preferred. This could be accomplished by catheterization, post-operatively in some cases, followed by continuous administration of the therapeutic agent. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the injections are administered. It is believed that higher doses may be achieved via perfusion, however.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, Remington's Pharmaceutical Sciences, 1990). Some variation in dosage will necessarily occur depending on the condition of the subject being treated. The person responsible for administration will, in any event, determine the appropriate dose for the individual subject.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability, and toxicity of the particular therapeutic substance.

Upon formulation, solutions will be administered in a manner compatible with the dosage formulation and in such amount as is therapeutically effective. The formulations are easily administered in a variety of dosage forms, such as the type of injectable solutions described above, but drug release capsules and the lilce can also be employed.

A. In Vitro, Ex Vivo, In Vivo Administration

As used herein, the term in vitro administration refers to manipulations performed on cells removed from an animal, including, but not limited to, cells in culture. The term ex vivo administration refers to cells which have been manipulated in vitro, and are subsequently administered to a living animal. The term in vivo administration includes all manipulations performed on cells within an animal.

In certain aspects of the present invention, the compositions may be administered either in vitro, ex vivo, or in vivo. U.S. Pat. Nos. 4,690,915 and 5,199,942, both incorporated herein by reference, disclose methods for ex vivo manipulation of blood mononuclear cells and bone marrow cells for use in therapeutic applications.

In vivo administration of the compositions of the present invention are also contemplated. Examples include, but are not limited to, transduction of bladder epithelium by administration of the transducing compositions of the present invention through intravesicle catheterization into the bladder, and transduction of liver cells by infusion of appropriate transducing compositions through the portal vein via a catheter. Additional examples include direct injection of tumors with the instant transducing compositions, and either intranasal or intratracheal (Dong, 1995) instillation of transducing compositions to effect transduction of lung cells.

### VI. KITS

In still further embodiments, the present invention concerns kits for use in therapy for cancer. The treatment kits will thus comprise, in suitable container means, a differentiation agent and an inhibitor or just the UC marker inhibitor.

The container means of the kits will generally include at least one vial, test tube, flask, bottle, syringe or other container means, into which the antibody or antigen may be placed, and preferably, suitably aliquoted. The kits of the present invention will also typically include a means for containing the antibody, antigen, and any other reagent containers in close confinement for commercial sale. Such containers may include injection or blow-molded plastic containers into which the desired vials are retained.

### VII. METHODS FOR SCREENING FOR MODULATORS

Differentiation agents discussed above modulate the expression of UC markers. In the present embodiment of the invention, the screening of other modulators of the expression of UC 28 and other UC proteins mentioned earlier in the description is contemplated.

The methods of screening may comprise assays that include random screening of large libraries of candidate substances; alternatively, the assays may be used to focus on particular classes of compounds selected with an eye towards structural attributes that are believed to make them more likely to function as a modulator of UC markers.

By function, it is meant that one may assay for a measurable effect on a candidate substance activity or inhibition of the expression of UC markers by the candidate substance. To identify a modulator of a UC marker, one generally will determine the activity or level of inhibition of a UC marker in the presence and absence of the candidate substance, wherein a modulator is defined as any substance that alters these characteristics. For example, a method generally comprises: (a) providing a candidate modulator; (b) admixing the candidate modulator with an isolated compound or cell, or a suitable experimental animal; (c) measuring one or more characteristics of the compound, cell or animal in step (b); and (d) comparing the characteristic measured in step (c) with the characteristic of the compound, cell or animal in the absence of said candidate modulator, wherein a difference between the measured characteristics indicates that said candidate modulator is, indeed, a modulator of the compound, cell or animal.

Assays may be conducted in cell free systems, in isolated cells, or in organisms including transgenic animals. It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate substance” refers to any molecule that may potentially modify the expression or activity of UC marker proteins. The candidate substance may inhibit or enhance expression of UC proteins or alter sensitivity of expression of UC proteins to inhibition. The candidate substance may be a protein or fragment thereof, a small molecule, or even a nucleic acid molecule. An example of pharmacological compounds will be compounds that are structurally related to UC proteins, or a substrate of UC proteins. Using lead compounds to help develop improved compounds is known as “rational drug design” and includes not only comparisons with known inhibitors and activators, but predictions relating to the structure of target molecules. An “inhibitor” is a molecule, which represses or prevents another molecule from engaging in a reaction. An “activator” is a compound that increases the activity of an enzyme or a protein that increases the production of a gene product in DNA transcription.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are well known to those of skill in the art. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on expression of UC proteins. Regardless of the type of inhibitor or activator identified by the present screening methods, the effect of the inhibition or activator by such a compound results in alteration in expression of UC proteins or susceptibility to inhibition of expression of UC proteins as compared to that observed in the absence of the added candidate substance.

The present invention provides methods of screening for a candidate substance that changes the expression of UC proteins. In these embodiments, the present invention is directed to a method for determining the ability of a candidate substance to change the expression of UC proteins, generally including the steps of: administering a candidate substance to the animal; and determining the ability of the candidate substance to reduce or enhance the expression of a UC protein.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

1. Cell Culture Methods

Cell lines, LNCap and C4-2B, were obtained from Dr. Leland Chung at the University of Texas MD Anderson Cancer Center (Thahnan et al., 1994)

Cell line MLC-SV40 was obtained from Dr. J.S. Rhim at National Cancer Institute.

LNCaP and C4-2B were cultured in T-Media, which is a combination of F-12 and DMEM. Vendor is Mediatech, Inc. 13884 Park Center Road, Herndon, Va. 20171 MLC-SV40 was cultured in Keratinocyte Serum Free Media with supplements. Vendor is Invitrogen Corp. 1600 Faraday Ave., P.O. Box 6482, Carlsbad, Calif. 92008.

Phenylbutyrate (4-Phenylbutyric Acid, Sodium Salt) was prepared at a 500 mM stock solution in PBS pH 7.2. Vendor is Triple Crown America, Inc. 13 N. 7^{th} Street, Perkasie, Pa. 18944.

2. Monoclonal Antibodies

The following monoclonal and polyclonal antibodies were prepared to SEQ ID NO:3 and SEQ ID NO:4 as shown below:

### UC28A Peptide (Amino Acids 54-74)

### UC28A-1 Rabbit Polyclonal Antibody

### Pab

### UC28A-3-1 G2 Mouse Monoclonal Antibody

### Mab

### UC28A-1-4 A3 Mouse Monoclonal Antibody

### Mab

### UC28A-3-3 Mouse Monoclonal Antibody

### Mab G10

### UC28A-1-4 C9 Mouse Monoclonal Antibody

### Mab

### UC28A-4-1 H5 Mouse Monoclonal Antibody

### Mab

### UC28C Peptide (Amino Acids 21-37)

### UC28C-1 Rabbit Polyclonal Antibody

### Pab

### UC28C-2-2 D2 Mouse Monoclonal Antibody

### Mab

### UC28C-1-1 A1 Mouse Monoclonal Antibody

### Mab

### UC28C-1-1 A2 Mouse Monoclonal Antibody

### Mab

### UC28C-3-1 F3 Mouse Monoclonal Antibody

### Mab

### UC28C-2-3 G2 Mouse Monoclonal Antibody

### Mab

3. Apoptosis Cell Culture Induction Experiments

Cells were grown to 70-80% confluence at 37° C. and 5% CO₂ in T-75 tissue culture plastic flasks. SPB stock solution was added to final concentrations of 0.5, 5.0, 10.0, and 25.0 mM in appropriate fresh media to the cell cultures. Cells were incubated at 37° C. and 5% CO₂ for 72 hours. Attached cells were then harvested using a solution of 0.05% trypsin and 0.53 mM EDTA (Invitrogen Corp.). Attached cells were then pooled with any the detached cells and washed with PBS pH 7.2. Cell numbers and percent viability were determined using Trypan blue dye exclusion and a hemacytometer. At the time of their use in an experiment the viability was usually about 85% for untreated cells and a range of about 15-70% for treated cells, with the lower viabilities found at higher SPB concentrations (Thompson C, 1995; Cory et al., 1998; van Engeland et al., 1996).

4. Flow Cytometry Procedures

a) Reagents for UC28, Annexin V, Fas, Bc1-2 and/or PI labeling:

VO1 Apoptosis Detection Kit contains: (1) Fluorocein-isothiocyante (FITC) conjugated AnnexinV [AnnexinV01] 1 ml (2) Propidium Iodide [Annex-Pi] 2.0 ml (3) 4× Binding Buffer [Annex-B] 20.0 ml. Vendor is CalTag Laboratories, Inc., 1849 Bayshore Boulevard #200 Burlingame, Calif. 94010.

UC28(C) is 2.14 mg/ml rabbit polyclonal aliquot directed against the inventors' UC28 patented biomarker developed by UCor R&D gene discovery program. The Antibody was produced against a twenty amino acid (AA) synthetic peptide from the predicted AA sequence (AA 21-37) UCor, Inc. 840 Research Parkway Oklahoma City, Okla. 73104.

Goat Anti-Rabbit CY5 polyclonal Fluorchrome was used as the secondary conjugate for UC28 C rabbit polyclonal antibody and a background control for UC28 C polyclonal. Vendor is Jackson ImmunoResearch Laboratories, Inc. 872 West Baltimore Pike, P.O. Box 9 West Grove, Pa. 19390.

MsIgG-FITC mouse conjugated-FITC was used for background control for AnnexinV01. Vendor is Becicman Coulter, Inc. Diagnostic Division, 11800 SW 147^{th} Ave. M/S 42-003, P.O. Box 169015, Miami, Floridia 33116-9015.

CD45 (FAS) IgG1 kappa—(R-phycoerythrin (RPE) is purified monoclonal antibody conjugated with R-phycoerythrin (RPE) that is specific for the FAS protein. Vendor is Dalco Corporation 6392 Via Real, Carpinteria, Calif. 93013 USA MsIgG1 IgG1 kappa R-phycoerythrin (RPE) conjugated goat anti-mouse immunoglobulins is used for (FAS) antibody negative control. Vendor is Dako Corporation 6392 Via Real, Carpinteria, Calif. 93013 USA.

Bc12 IgG1 kappa-FITC isomer 1 is a purified monoclonal mouse specific for Bc1-2 protein. Vendor is Dalco Corporation 6392 Via Real, Carpinteria, Calif. 93013 USA.

MsIgG1 kappa-FITC isomer 1 is a purified isotype specific control for Bc1-2 monoclonal antibody. Vendor is Dako Corporation 6392 Via Real, Carpinteria, Calif. 93013 USA.

Cytofix/Cytoperm™ ICit enables fixation and permeabilization of cells prior to staining with fluorochrome-conjugated antibodies. Vendor is BD-PharMingen, 10975 Torreyana Road, San Diego, Calif. 92121-1106.

b) Flow Cytometer Set-Up:

A Coulter Elite engineered with a triple laser system was used to analyze all samples. Coulter control beads were used to align lasers for the dual label testing of cell samples. Laser alignment was required for both the Argon (488 nm line) laser and HeNe (633 nm line) laser. These alignments were performed prior to all measurements with the Argon laser (488 nm line) using fluorophores for FITC, RPE, PI and the HeNe laser (633 nm line) for the Cy5 fluorophores being used for the experiments described below. The histographs, voltage settings, gates, color compensation, and pressures were established prior to all sample processing.

c) Protocol for FCM AnnexinV and.Propidium Iodide (PI) Labeling:
(i) The five 12×75 mm polystyrene tubes were labelled as follows: 1) No Antibody 2) MsIgG-FITC 3) AnnexinV-FITC 4) PI only 5) AnnexinV with PI.
(ii) A volume of 1.5×10⁶ tissue culture cells were aliquoted into each 12×75 mm polystyrene tube and cells were washed by adding 3 ml of cold PBS (phosphate buffered saline) pH7.3 to tube. Cells were pelleted by centrifugation at 500 g×5 minutes. The tubes were removed from centrifuge and supernatant was removed by aspirating without disturbing cells.
(iii) The cells were resuspended in 1× Binding Buffer (made from 4× solution from AnnexinVOl kit). The cells were vortexed gently and placed for 15 minutes at ambient temperature (20-25° C.) in the dark.
(iv) The tubes were removed from the dark and 100111 (0.5×10⁶) cells were alquoted into each of the 12×75 mm polystyrene tubes.
(v) A volume of 5 μl of AnnexinV, 5 μl MsIgG-FITC, and 101.11 of PI were added to the appropriate labeled tubes (See #1 above for labeled tubes).
(vi) The tubes were gently vortexed tubes and incubated for 15 minutes at ambient temperature (20-25° C.) in the dark.
(vii) A volume of 400 μl of AnnexinV kit Binding Buffer was added to each tube.
(viii) Analysis by Flow Cytometry (FCM) for AnnexinV and PI activity was carried out as soon as possible.

5. Protocol For Dual Labeling with UC28 C Polyclonal and FAS Monoclonal or UC28 C Polyclonal and Ben Monoclonal

(a) Tissue culture cells (MLC-SV40, LNCaP and C 4-2B) were aliquoted at concentration of 1×10⁶ per teach 12×75 mm polystyrene tube. Label tubes as follows:
(i) No Antibody
(ii) Secondary GAR-CY5 polyclonal=(control for UC28 CIDGARCY5 polyclonal) vs MsIgG1-RPE (control for FAS-RPE monoclonal)
(iii) UC28 C-IDGARCY5 polyclonal vs FAS (CD45)—RPE monoclonal
(iv) SecondaryGAR-CY5 polyclonal=(control for UC28 C-IDGARCY5 polyclonal) vs MsIgG1-FITC (control for Bc12-FITC monoclonal)
(v) UC28 C-IDGARCY5 polyclonal vs Bc12-FITC monoclonal
(vi) UC28 C-IDGARCY5 Polyclonal=(single label for checking instrument after setting Coulter Elite flow cytometer for dual analysis using the three different fluorchromes.)
(vii) FAS (CD34)—RPE monoclonal=(single label for checking instrument after setting Coulter Elite flow cytometer for dual analysis using the three different fluorchromes.)
(viii) Bc12-FITC monoclonal=(single label for checking instrument after setting Coulter Elite flow cytometer for dual analysis using the three different fluorchromes.)

(b) All tubes were centrifuged at 500 g for 5 minutes using a Jouan centrifuge. All tubes were removed and supernatant aspirated carefully from pelleted cells being careful not to disturb pellet.

(c) A volume of 100 μl of UC28 C rabbit polyclonal unconjugated antibody was added to the appropriately labeled tubes. All other tubes received 100 μl PBS (pH7.3) and all tubes were incubated at room temp in the dark for 1 hour.

(d) After incubation, all tubes received 2 ml of PBS pH7.3 and were centrifuged at 500 g for 5 minutes. Next, all tubes were removed from centrifuge and supernatants were removed carefully not to disturb pelleted cells. A PBS wash procedure was performed times 2.

(e) Secondary goat anti-rabbit Cy5 labeled antibody was added in PBS pH7.3 to appropriately labeled tubes. (NOTE: Prepare enough secondary CY5 labeled antibody to add 100 μl for each test being done by adding 10 μl of GAR-CY5 to 90 μl of PBS pH7.3).

(f) Secondary goat anti-rabbit Cy5 was incubated in the dark at ambient temperature for 30-45 minutes. (NOTE: During the incubation period a second biomarker can be added if it is a surface expressed antigen such as CD45 (FAS)—RPE).

(g) To the CD45 (FAS) labeled tubes 10 μl of the monoclonal dilution was added directly to samples and mixed. Also the isotype control for the specific monoclonal isotype was prepared by adding 141 of MsIgG1-RPE (monoclonal isotype) to 90 μl of PBS pH7.3 and then 100 μl solution was added to the appropriate labeled control tubes. All tubes were placed back in the dark to continue incubation for 30 minutes.

(h) All tubes were removed from centrifuge and cells were washed twice as in step “c” above.

(i) Next, the cells were fixed and permeabilized by adding 500 μl of CytoFix/Cytoperm solution. The solution was vortexed vortexed gently and incubated at room temperature in the dark for 20 minutes. The sample was removed from dark and centrifuged. The supernatant was removed from pelleted cells and mixed gently.

(j) A volume of 2 ml Perm/Wash® solution wasa added to all appropriate labeled tubes and centrifuge for five minutes at 500×g. The tubes were removed from centrifuge and supernatant was aspirated from cell being careful not to disturb cells.

(k) The cell pellets were resuspended in 100 μl Penn/Wash® solution containing 10 μl each of Bc12-FITC—conjugated monoclonal antibody and were added to the appropriate labeled tubes. At this time the MsIgG1-FITC-conjugated isotype control was added to the appropriate labeled tubes by adding 10p. 1 of conjugated isotype control to the appropriate labeled tubes. All tubes were incubated in the dark for 30-45 minutes.

(l) The tubes were removed from the dark and cells were resuspended in 2 ml of PenniWash® solution and were centrifuged using Jouan centrifuge at 500 g for 5 minutes to pellet cells. The supernatant was aspirated carefully from all pellet cells and mixed gently.

(m) A volume of 1 ml of 0.5% paraformaldehyde was added while mixing to each tube and cap. All tubes were placed into 2°-8° C. refrigerator in the dark until FCM can be performed.

Note: These samples can be held for 3 days at 2°-8° C. before analysis if needed. Also, this method can be used for viewing and photographing of the same cell preparations used for FCM by adding 10-15 drops (by using pasture pipette) into 12×75 mm tube and adding two drops of DAKOR fluorescent mounting medium (Cat. No. 53023) to each tube. For photogjaphy, mix the cell preparation solution and remove several drops of the mixture from each tube and place on individual 1×3 inch microscope glass slides. Coverslip each slide and place in a slide holder in the dark at 2°-4° C. until ready to view and photograph. Use a fluorescent microscope such as the Leitz with the appropriate excitation filters to view the fluorchromes.

### Introduction to Examples 2-7

Three prostate cell lines were utilized to evaluate the effects of sodium phenylbutryate (SPB) treatment on induction of UC28 protein expression and apoptosis. The cell lines were MLC-SV40, a virally immortalized normal epithelial cell line, LNCaP, a malignant prostate epithelial cell line derived from the lymph node of a patient with prostate cancer, and the C 4-2B variant of LNCaP, which is a bone metastatic variant of the LNCaP parent epithelial cell line (Ng et al., 1997). SPB has been employed in clinical trials to treat cancer based upon its cellular differentiating and cell growth inhibitory activities (Ng et al., 2000). SPB has also been specifically applied to the treatment of prostate cancer patients (Carducci M A et al., 1996).

The UC28 gene was discovered using arbitrarily primed RNA fingerprinting methodology to human normal benign and cancer tissues (U.S. Pat. No. 5,882,864) and subsequently cloned (U.S. Pat. No. 6,171,796). Highly specific rabbit polyclonal antibodies were prepared against synthetic peptides of the UC28 protein and used for assessment of protein expression and RT-PCR was employed to assess the mRNA expression in human cell lines as well as human tumors (An et al., 2000). These studies of the UC28 gene expression at both the mRNA and protein levels in vivo clearly indicated an up regulation of the gene and the protein it codes for in prostate, breast, and bladder cancer (An et al., 2000).

### Example 2

### Effect of SPB on UC28 Gene Expression

The experiment was conducted to determine the impact of SPB on UC28 gene expression as well as its relationship to biochemical alterations of the apoptosis pathway in vitro. UC28 protein was localized on a membrane using rabbit polyclonal antibody produced against a UC28 synthetic peptide and visualized fluorescent confocal imaging technology. C4-2B is a sub-clone of the human LNCaP cell line developed in Dr. Leland Chung's laboratory (Thalmann et al., 1994), which is a bone metastatic cell line. The cells have been labeled with red fluorescent lipid-specific membrane stain (DiD, 1,1′-dioctadecyl-3,3,3′,3′-tetramethylindodicarbocyanine) and a green/yellow fluorescent labeled antibody directed at UC28 prostate gene-coded protein (An et al., 2000). It was found that a significant portion of the UC-28 protein localizes to the cell membrane of prostate cells.

### Example 3

### Induction by SPB

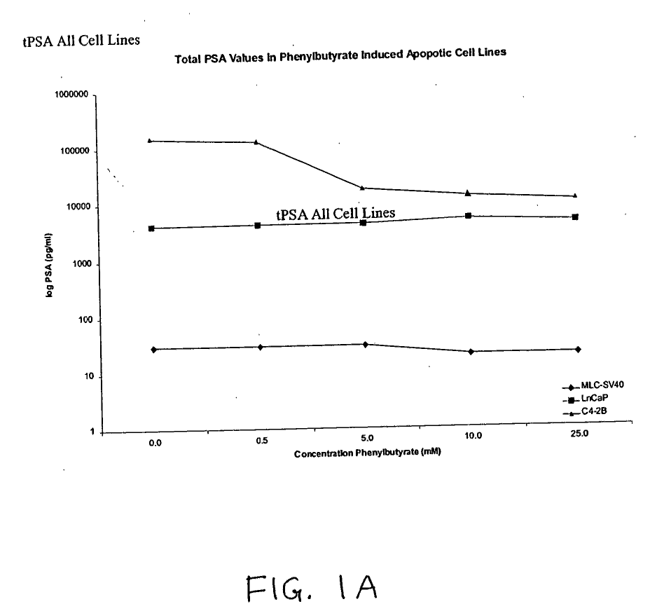

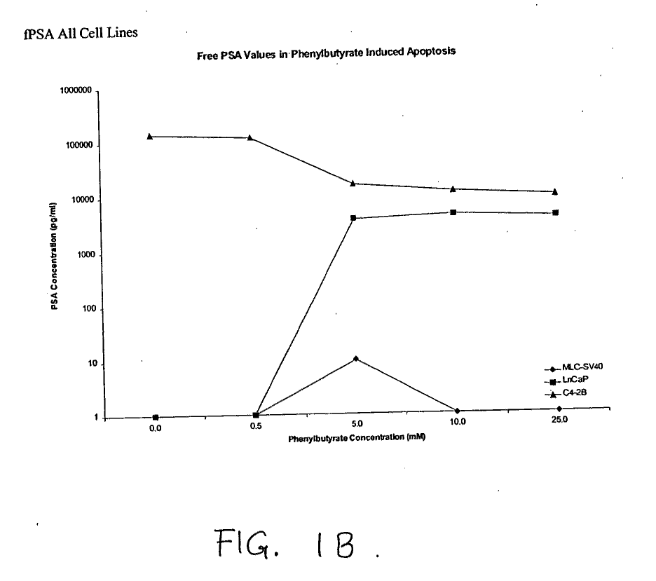

The secretion of soluble tPSA and fPSA under the influence of sodium phenylbutryate (SPB) treatment demonstrated first, differential expression of tPSA in the three different cell lines (FIGS. 1A and 1B). Also, both tPSA and fPSA production were down-regulated in the C4-2B metastatic cell line. URCO28 expression in the LNCap and C4-2B lines remained elevated when treated with SPB. The data confirms the biological differences in these three cell lines and provides a stronger basis upon which to assess the behavior of the UC28 gene under the same SPB treatment conditions. For both of these experiments, supernatants were collected from the same experiments as in Example 1; cells were collected and assayed for apoptosis as well as for UC28 antigen expression using antibodies to SEQ ID NO:4.

### Example 4

### Dose Response Kinetics of UC28 Expression

The dose-response kinetics of UC28 protein expression using flow cytometry and the same antibody to UC28 and three prostate cell lines that differ in their malignant potential is demonstrated in FIG. 1 The LNCaP and C4-2B cell lines are dramatically over expressing the UC28 membrane protein at the various doses of 0.5 to 25 mM SPB, however, the MLC-SV40 immortalized normal cells do not over express UC28 membrane protein.

### Example 5

### Correlation of Induction of UC28 by SPB with Induction of Apoptosis

UC28 and Annexin V were co-labeled in the three cell lines to assess the induction of UC28 by SPB and its correlation to the induction of apoptosis. Table 5 shows that apoptosis is induced in all three cell types but that UC28 is only up regulated in cancer cell lines (LNCaP and C4-2B) and not in the immortalized normal MLC-SV40 cell type. It may be noted that in previously published experiments, AnnexinV and PI were run in combination with several prostate cancer cell lines, one of which was LNCaP, but not with C4-2B or MLC-SV40. It was determined that they produced similar dose-response kinetics when exposed to 0.5-25 mM of SPB for 72 hours (Ng et al., 1989). That is, as the dose of SPB increased, so did the membrane perturbation events measured by these two methods.

TABLE 5

| | | | |
|---|---|---|---|
| mM | | Total | Total |
| SPB | Primary Combination | UC28C | AnnexV |

Dual Label: UC28 C/Ann xinV in ML-SV40 Prostat Cell Line

| | | | |
|---|---|---|---|
| 0 | UC28-GAR-CY5/AnnexV-FITC | 8.0% | 0.8% |
| 0.5 | UC28-GAR-CY5/AnnexV-FITC | 8.6% | 11.4% |
| 5.0 | UC28-GAR-CY5/AnnexV-FITC | 6.8% | 90.2% |
| 10 | UC28-GAR-CY5/AnnexV-FITC | 6.8% | 95.7% |
| 25 | UC28-GAR-CY5/AnnexV-FITC | 9.3% | 90.7% |

Dual Label: UC28/AnnexinV in LNCaP Prostate Cell Line

| | | | |
|---|---|---|---|
| 0 | UC28-GAR-CY5/AnnexV-FITC | 11.5% | 4.5% |
| 0.5 | UC28-GAR-CY5/AnnexV-FITC | 28.0% | 10.6% |
| 5.0 | UC28-GAR-CY5/AnnexV-FITC | 55.8% | 44.7% |
| 10 | UC28-GAR-CY5/AnnexV-FITC | 66.4% | 33.6% |
| 25 | UC28-GAR-CY5/AnnexV-FITC | 67.4% | 89.2% |

Dual Label: UC28 C/AnnexinV in C4-2B Prostate Cell Line

| | | | |
|---|---|---|---|
| 0 | UC28-GAR-CY5/AnnexV-FITC | 33.7% | 14.4% |
| 0.5 | UC28-GAR-CY5/AnnexV-FITC | 29.8% | 41.1% |
| 5.0 | UC28-GAR-CY5/AnnexV-FITC | 81.8% | 94.0% |
| 10 | UC28-GAR-CY5/AnnexV-FITC | 88.0% | 80.0% |
| 25 | UC28-GAR-CY5/AnnexV-FITC | 96.5% | 82.3% |

### Example 6

### Coordinate Expression of Bc1-2 with UC28

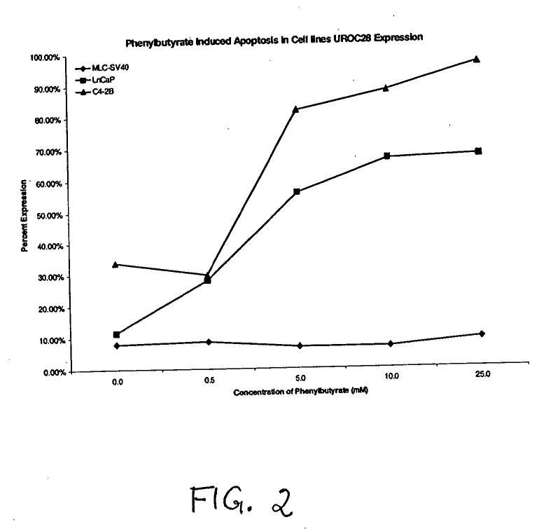

An additional set of experiments were conducted to demonstrate that bc1-2, another important apoptosis pathway member was coordinately expressed with UC28 in a subset of apoptotic cells at doses shown in Table 6 and FIG. 2 to induce apoptosis and UC28. UC28 and Bc1-2 were co-labeled in LNCaP cell line. It was found that a significant percentage of cells expressed only UC28.

TABLE 6
Dual Label: UROC28/Bcl-2 in LNCaP Prostate Cell Line

| mM | | Total | Total |
|---|---|---|---|
| SPB | Primary Combination | UC2BC | AnnexV |
| 0 | UROC28/Bcl-2 | 16.2% | 11.8% |
| 5.0 | UROC28/Bcl-2 | 87.7% | 23.8% |
| 10 | UROC28/Bcl-2 | 83.0% | 15.6% |

### Example 7

### Coordinate Expression of UC28 and Bc1-2 and Fas

The coordinate expression of UC28 and both Bc1-2 and Fas proteins in triple cell label experiments evaluated using Flow cytometry and the C4-2B metastatic cell line variant of LNCaP were assessed. Table 7 demonstrates in the C4-2B metastatic LNCaP cell line variant that although UC28 is significantly up regulated by SPB, the Bc1-2 and Fas proetin expression are both markedly depressed by SPB. This is in contrast to the results in Table 6 above for Bc1-2, where UC28 and Bc1-2 are decreased but in many cells remain coordinately expressed.

TABLE 7

| | | | |
|---|---|---|---|
| mM SPB | Primary Combination | Total UC2BC | Total Fas |

UROC28/Bcl-2 in C4-2B Metastatic Prostate Cell Line

| | | | |
|---|---|---|---|
| 0 | UROC28/Bcl-2 | 20.9% | 95.0% |
| 5.0 | UROC28/Bcl-2 | 96.0% | 5.4% |
| 10 | UROC28/Bcl-2 | 93.9% | 0.3% |

UROC28/Fas in C4-2B Metastatic Prostate Cell Line

| | | | |
|---|---|---|---|
| 0 | UROC28/Bcl-2 | 27.0% | 64.2% |
| 5.0 | UROC28/Bcl-2 | 96.4% | 3.6% |
| 10 | UROC28/Bcl-2 | 94.0% | 6.1% |

All of the COMPOSITIONS and METHODS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS and METHODS and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following literature citations as well as those cited above are incorporated in pertinent part by reference herein for the reasons cited in the above text.
U.S. Pat. No. 3,791,932
U.S. Pat. No. 3,817,837
U.S. Pat. No. 3,850,752
U.S. Pat. No. 3,939,350
U.S. Pat. No. 3,949,064
U.S. Pat. No. 3,996,345
U.S. Pat. No. 4,174,384
U.S. Pat. No. 4,196,265
U.S. Pat. No. 4,275,149
U.S. Pat. No. 4,277,437
U.S. Pat. No. 4,340,535
U.S. Pat. No. 4,366,241
U.S. Pat. No. 4,472,509
U.S. Pat. No. 4,690,915
U.S. Pat. No. 4,659,774
U.S. Pat. No. 4,682,195
U.S. Pat. No. 4,683,202
U.S. Pat. No. 4,797,368
U.S. Pat. No. 4,816,571
U.S. Pat. No. 4,879,236
U.S. Pat. No. 4,938,948
U.S. Pat. No. 4,959,463
U.S. Pat. No. 5,021,236
U.S. Pat. No. 5,139,941
U.S. Pat. No. 5,141,813
U.S. Pat. No. 5,199,942
U.S. Pat. No. 5,264,566
U.S. Pat. No. 5,359,046
U.S. Pat. No. 5,428,148
U.S. Pat. No. 5,554,744
U.S. Pat. No. 5,574,146
U.S. Pat. No. 5,602,244
U.S. Pat. No. 5,620,896
U.S. Pat. No. 5,645,897
U.S. Pat. No. 5,693,762
U.S. Pat. No. 5,705,629
U.S. Pat. No. 5,861,155
U.S. Pat. No. 5,871,986
U.S. Pat. No. 5,882,864
U.S. Pat. No. 5,958,895
U.S. Pat. No. 5,976,546
U.S. Pat. No. 6,020,192
U.S. Pat. No. 6,054,297
U.S. Pat. No. 6,099,842
U.S. Pat. No. 6,121,044
U.S. Pat. No. 6,171,796
U.S. Pat. No. 6,210,662
U.S. Pat. No. 6,218,529
JP-A 6-179622
JP-A6-192073
JP-A6-256181
JP-A 6-305955
JP-A 6-316520
JP-A 7-206765
JP-A 7-258100
Aksentijevich et al., Hum Gene Ther, 7(9):1111-1122, 1996.
An et al., Cancer Res. 60: 7014-7020, 2000.
An et al., Proc. Annu Meet Am Assoc Cancer Res., 38:A2812, 1997.
An et al., AACR/EROTC/NCl Chemoprevention Meeting, September 1999.
Antibodies: A Laboratory Manual, Cold Spring Harbor Laboratory, Cold Spring Harbor Press,
Cold Spring Harbor, N.Y., 1988.
Atherton et al., Biol Reprod, 32(1):155-171, 1985.
Ausubel et al., In: Current Protocols in Molecular Biology, John, Wiley & Sons, Inc, New York, 1994.
Baichwal and Sugden, In: Gene Transfer, Kucherlapati R, ed., New York, Plenum Press, pp. 117148, 1986.
Bangham et al., J. Mol. Biol. 13: 238-252, 1965.
Beduschi et al., Urology, 51:861-872, 1998.
Bedzyk et al., J. Biol. Chem., 265(30):18615-18620, 1990.
Berberian et al., Science, 261(5128):1588-1591, 1993.
Berchem et al., Cancer Research; 55:735-738, 1995.
Berges et al., Proc Natl Acad Sci USA, 90:8910-8914, 1993.
Bittner et al., Methods in Enzymol, 153:516-544, 1987.
Borner et al., Cancer Res, 55:2122-2128, 1995.
Butler et al., Cancer Res., 60(18):5165-5170, 2000.
Caley et al., J Virol, 71(4):3031-3038, 1997.
Campbell et al., Br J Cancer, 77:739-744, 1998.
Candido et al., Cell 14: 105-113, 1978.
Candido.et al., Cell, 14:105-113, 1978.
Capaldi et al., Biochem. Biophys. Res. Comm., 76:425, 1977.
Cardillo et al., J Urol, 158:212-216, 1997.
Carducci et al., Clin Cancer Res, 2(2):379-87, 1996.
Carducci et al. Anticancer Res. 17: 3972, 1997.
Carroll et al., Prostate, 23:123-134, 1993.
Castaign et al., Blood, 76:1704-1709, 1990.
Chang et al., Hepatology, 14:124 A, 1991.
Chaudhary et al., Proc. Natl. Acad. Sci. USA, 87(3):1066-1070, 1990.
Chresta et al., Cancer Res, 56:1834-1841, 1996.
Clark et al., Hum. Gene Ther., 6(10):1329-1341, 1995.
Cleary et al., J. Biol. Chem., 269(29):18747-9, 1994.
Coffin et al., In: Virology, Fields et al., eds., Raven Press, New York, pp. 1437-1500, 1990.
Cohen et al., Anticancer Res., 19(6B):4999-5005, 1999.
Colberre-Garapin et al., J. Mol. Biol., 150:1, 1981.
Colombel et al., Am J Pathol, 143:390-400, 1993.
Cory et al., Biochimica et Biophysica Acta 1377: R25—R44, 1998.
Coupar et al., Gene, 68:1-10, 1988.
Dahiya et al., Biochem Mol Biol Int, 32(1):1-12 1994
Danesi et al. Mol Pharmacol 47:1106-1111, 1995.
D'Anna et al., Biochemistry, 19:2656-2671; 1980.
Deamer and Uster, “Liposome Preparation: Methods and Mechanisms,” In: Liposomes M. Ostro, ed., 1983.
Dholakia et al., J. Biol. Chem., 264(34):20638-20642, 1989.
Dong et al., Science, 268: 884-886, 1995.
Dumont et al., J Immunol, 152:992-1003, 1994.
EP 0273085
EP 266,032
Faller D et al., Curr Opin Hematol, 2:109-117, 1995.
Feigner et at, Proc Nall Acad Sci USA, 84(21):7413-7, 1987.
Figg et al., Anticancer Drugs; 5:336-342, 1994.
Finnin et al., Nature, 401(6749):188-193, 1999.
Flotte et al., Gene Ther, 2(1):29-37, 1995.
Flotte et al., Proc Nall Acad Sci USA, 90(22):10613-7, 1993.
Fraley et al., Proc. Nat'l Acad. Sci. USA, 76:3348-3352, 1979.
Friedmann, Science, 244:1275-1281, 1989.
Gabizon et al., Cancer Res, 50(19):6371-8, 1996.
Ghosh and Bachhawat, In: Liver Diseases, Targeted Diagnosis and Therapy Using Specific Receptors and Ligands. Wu et al., eds., Marcel Dekker, New York, pp. 87-104, 1991.
Ghosh-Choudhury et al., EMBO J., 6:1733-1739, 1987.
Goding, In: Monoclonal Antibodies: Principles and Practice, 2d ed., Orlando, Fla., Academic
Press, pp. 60-61, 65-66, 71-74, 1986.
Goillot et al., Proc Natl Acad Sci USA, 94:3302-3307, 1997.
Gomez-Foix et al., J. Biol. Chem., 267:25129-25134, 1992.
Gorospe et al., Cell Growth Differ. 7: 1609-1615, 1996.
Graham and Prevec, Biotechnology, 20:363-390, 1992.
Gregoriadis, In: Drug carriers in biology and medicine, pp. 287-341, 1979.
Grunhaus and Horwitz, Seminar in Virology, 3:237-252, 1992.
Hague et al. Int J Cancer; 55:498-505, 1993.
Halicka et al., Proc. Am. Soc. Clin. Oncol., 32^{nd} Annual Meet, 1996.
Hermonat and Muzycska, Proc. Nat. Acad. Sci. USA, 81:6466-6470, 1984.
Hersdorffer et al., DNA Cell Biol., 9:713-723, 1990.
Herz and Gerard, Proc. Nat'l Acad. Sci. USA, 90:2812-2816, 1993.
Horwich, et al., Virol., 64:642-650, 1990.
Huang and Pardee, Mol. Med., 6(10):849-866, 2000.
Huang et al., Blood, 72:567-572, 1988.
Huang et al., Urology, 51:346-351, 1998.
Itoh et al., Cell, 66:233-243, 1991.
Johnson et al., In: Biotechnology and pharmacy, Pezzuto et al., Eds., Chapman and Hall, New York, 1993.
Kamesaki et al., Cancer Res, 53:4251-4256, 1993.
Kaneda et al., Science, 243:375-378, 1989.
Kang et al., Science, 240(4855):1034-6, 1988.
Kato et al., J. Biol. Chem., 266:3361-3364, 1991.
Khatoon et al., Ann Neurol, 26(2):210-5, 1989.
Kimura and Yamamoto, Cell Biochem Funct, 15:81-86, 1997.
King et al., J Biol Chem, 264(17):10210-10218, 1989.
Kohler et al., Methods Enzymol, 178:3-35, 1989.
Korsmeyer et al., Semin Cancer Biol, 4:327-332, 1993.
Kotin et al., Proc Natl Acad Sci USA, 1990 March; 87(6):2211-5, 1990.
Krajewska et al., Am J Pathol, 148:1567-1576, 1996.
Kyprianou et al., Int J Cancer, 70:341-348, 1997.
Kyte and Doolittle, J. Mol. Mot, 157(1):105-132, 1982.
LaFace et al., Virology, 162(2):483-486, 1988.
Laughlin et al., J Virol, 60(2):515-524, 1986.
Le Gal La Salle et al., Science, 259:988-990, 1993.
Lea et al., Anticancer Res. 18: 2717-2722, 1998.
Lebkowski et al., Mol Cell Biol, 8(10):3988-3996, 1988.
Lenert et al., Science, 248(4963):1639-43, 1990.
Levrero et al., Gene, 101:195-202, 1991.
Lippman and Davies, Cancer Chemother Biol Response Modif, 17:349-362, 1997.
Lowy et al., Cell, 22:817, 1980.
Maestri et at, N Engl J Med; 335:855-859, 1996.
Mann et al., Cell, 33:153-159, 1983.
Markowitz et al., J. Virol., 62:1120-1124, 1988.
McCarty et al., J Virol, 65(6):2936-45, 1991.
McConkey et al., Cancer Res, 56:5594-5599, 1996.
McDonnell et al., Cancer Res, 152:6940-6944, 1992.
McLaughlin et al., J Virol, 62(6):1963-1973, 1988.
Melchior et al., Int J Oncol, 14(3):501-508 1999).
Melchoir et al., Int. J. Oncol. 14: 17174-17179, 1999.
Mulligan et al., Proc. Nat'l Acad. Sci. USA, 78:2072, 1981.
Muzyczka, Curr Top Microbiol Immunol, 158:97-129, 1992.
Nakamura et al., In: Handbook of Experimental Immunology (4th Ed.), Weir et al., (eds), 1:27, Blackwell Scientific Publ., Oxford, 1987.
Nakano et al., J Biochem; 272:22199-22206, 1997.
Ng et al., Proc Am Assoc Cancer Res, 39:571, 1998.
Ng et al., Proc Am Assoc Cancer Res, 39:A1790, 1998.
Ng et al., Proc Am Assoc Cancer Res, 39:A3882, 1998.
Ng et al., Anal. Quant. Cytol. Histol., 22(1): 45-54, 2000.
Ng et al., Nuc. Acids Res., 17:601, 1989.
Ng et al., Proc. Annu. Meet Am. Assoc. Cancer Res., 38: A615, 1997.
Ng et al., Proc Am Assoc Cancer Res, 38:92, 1997.
Nicolas and Rubinstein, In: Vectors: A survey of molecular cloning vectors and their uses, Stoneham: Butterworth, pp. 494-513, 1988.
Nicolau and Sene, Biochim. Biophys. Acta, 721:185-190, 1982.
Nicolau et al., Methods Enzymol., 149:157-176, 1987.
O'Hare et al., Proc. Nat'l Acad. Sci. USA, 78:1527, 1981.
Ohi et al., Gene, 89(2):279-282, 1990.
Olsson et al., Cancer Res. 43:5862-5867, 1983.
Oltvai et al., Cell, 74:609-619, 1993.
Ouyang et al., Clinical Cancer Res, 4:1071-1074, 1998.
Owens and Hardy, Biochem Biophys Res Commun, 142(3):964-71, 1987.
Paskind et al., Virology, 67:242-248, 1975.
PCT Application WO 89/00999
Potter and Haley, Methods Enzymol, 91:613-633, 1983.
Qiao et al., Biochem. Biophys. Res. Comm., 201:581-588, 1994.
Raffo et al., Cancer Res; 55:4438-4445; 1995.
Ragot et al., Nature, 361:647-650, 1993.
Ravindranath and Morton, Intern. Rev. Immunol., 7: 303-329, 1991.
Reed, Curr Opin Oncol, 1995; 7:541-546, 1995.
Remington's Pharmaceutical Sciences, 18th Edition, Chapter 61, pages 1289-1329, 1990.
Renan, Radiother. Oncol., 19:197-218, 1990.
Ribas de Pouplana and Fothergill-Gilmore, Biochemistry, 33:7047-7055, 1994.
Rich et al., Hum. Gene Ther., 4:461-476, 1993.
Richon et al., Proc. Natl. Acad. Sci. 95: 3003-3007, 1998.
Ridgeway, In: Vectors: A survey of molecular cloning vectors and their uses, Rodriguez et al., (Eds.), Stoneham:Butterworth, pp. 467-492, 1988.
Rieber and Rieber, Cell Growth Dyf, 5:1339-1346, 1994.
Rokhlin et al., Cancer Res, 57:1758-1768, 1997.
Rosenberg et al., Ann Surg. 210(4):474-548, 1989
Rosenberg et al., N. Engl. J. Med., 319:1676, 1988.
Rosenfeld et al., Cell, 68:143-155, 1992.
Rosenfeld et al., Science, 252:431-434, 1991.
Roux et al., Proc. Nat'l Acad. Sci. USA, 86:9079-9083, 1989.
Rovere et al., Arthritis Rheum., 1999 July; 42(7):1412-2140, 1999
Rubenstein et al., Am J Respir Crit. Care Med, 157:484-490, 1998.
Sambrook et at, In: Molecular cloning, Cold Spring Harbor Laboratory Press, Cold Spring Harbor, N.Y., 1989.
Sambucetti et al., J. Biol. Chem. 274: 34940-34947, 1999.
Samid et al., Adv Exp Med Biol, 400A:501-5, 1997
Samid et al., Blood, 80(6):1576-81, 1992.
Samulski et al., EMBO J, 10:3941-3950, 1991.
Samulski et al., J. Virol, 63:3822-3828, 1989.
Santerre et al., Gene, 30: 147-156, 1984.
Sasso et al., J. Immunol., 142:2778-2783, 1989.
Shelling and Smith, Gene Therapy, 1:165-169, 1994.
Shorki et al., J. Immunol., 146:936-940, 1991.
Silvermann et al., J. Clin. Invest., 96:417-426, 1995.
Soldatenkov et al., Cell Death and Differentiation, 307-312, 1998.
Solodin et al., Biochemistry, 34(41):13537-13544, 1995.
Steinman et al., Hum Immunol, 60(7):562-567, 1999.
Stratford-Perricaudet and Perricaudet, In: Human Gene Transfer, Cohen-Haguenauer et al., eds., John Libbey Eurotext, France, pp. 51-61, 1991.
Stratford-Perricaudet et al., Hum. Gene. Ther., 1:241-256, 1990.
Suda and Nagata, J. Exp. Med., 179:873-878, 1994.
Sun and Cohen, Gene, 137:127-132, 1993.
Suzuki et al., Oncogene, 13:31-37, 1996.
Szoka and Papahadjopoulos, Proc. Natl. Acad. Sci. USA, 75: 4194-4198, 1978.
Szybalska et al., Proc. Nat'l Acad. Sci. USA, 48:2026, 1962.
Temin, In: Gene Transfer, New York: Plenum Press, pp. 149-188, 1986.
Thalman et al., Cancer Research. 54: 2566-2851, 1994.
Thalmann et al. Cancer Res; 54:2577-2581, 1994.
Thierry et al., Proc Natl Acad Sci USA. 92(21):9742-9746, 1995.
Thompson, Science 267: 1456-1462, 1995.
Thompson, Science, 267:1456-1462, 1995.
Top et al., J. Infect. Dis., 124:155-160, 1971.
Tratschin et al., Ma Cell. Biol., 4:2072-2081, 1984.
Tratschin et al., Mol. Cell. Biol., 5:3258-3260, 1985. van Engeland et al., Cytometiy, 24:131-139, 1996.
Vaziri et al., Cell Growth & Differentiation, 9:465-474, 1998.
Veltri et al., Pro. Annu. Meeting Amer. Assoc. of Cancer Res., 39:A3882, 1998.
Wagner et al., Science, 260:1510-1513, 1993.
Walsh et al., J. Clin. Invest, 94:1440-1448, 1994.
Warren et al., New Engl. J. Med. 324:1385-1393, 1991.
Wei et al., Gene Therapy, 1:261-268, 1994.
Wigler et al., Cell, 11:223, 1977.
Wigler et al., Proc. Nat'l Acad. Sci. USA, 77:3567, 1980.
Wintersberger et al., J Cell Biochem, 21:239-247, 1983.
Wong et al., Gene, 10:87-94, 1980.
Wood et al., Proc. Am Assoc Cancer Res, 35:A2404, 1994.
Wood et al., Proc Am Assoc Cancer Res, 36:A3831, 1995.
Wu et al., Int. J. Cancer; 57:406-412; 1994.
Yang and Huang, Gene Therapy, 4 (9):950-960, 1997.
Yang et al., Proc. Natl. Acad. Sci. USA, 91:4407-4411, 1994.
Yoder et al., Blood, 82 (Supp.): 1:347 A, 1994.
Zhou et al., Exp. Hematol, 21:928-933, 1993.
Zhou et al., J. Exp.Med., 179:1867-1875, 1994.
Zhu et al., Science. 261(5118):209-211, 1993.

1-72. (canceled)

73. A method of detecting malignant prostate cancer in a sample obtained from a subject, the method comprising the steps of:
(a) measuring an amount of UC28 expression in the sample; and
(b) indicating that malignant prostate cancer is present in the sample if the level of UC28 expression in the sample is elevated as compared to the level of UC28 expression in a noncancerous epithelial reference sample.

74. The method of claim 73, wherein the malignant prostate cancer comprises bone metastatic prostate cancer.

75. The method of claim 73, wherein the step of measuring the level of UC28 expression comprises contacting the sample with a UC28 antibody that specifically binds to UC28 protein, and measuring the amount of the UC28 antibody bound to the sample.

76. The method of claim 73, wherein the step of measuring the level of UC28 expression comprises isolating ribonucleic acid (RNA) from the sample, and measuring the amount of UC28 RNA present.

77-104. (canceled)

105. A method of treating a subject having malignant prostate cancer comprising:
administering a UC28 targeted therapy or a differentiation agent to the subject if the level of UC28 expression subject's cancer is elevated compared to the level of UC28 expression in a noncancerous epithelial reference.

106. The method of claim 105, wherein the malignant prostate cancer comprises bone metastatic prostate cancer.

107. The method of claim 123, wherein the step of measuring the level of UC28 expression comprises contacting the sample with a UC28 antibody that specifically binds to UC28 protein, and measuring the amount of the UC28 antibody bound to the sample.

108. The method of claim 123, wherein the step of measuring the level of UC28 expression comprises isolating ribonucleic acid (RNA) from the sample, and measuring the amount of UC28 RNA present.

109-112. (canceled)

113. The method of claim 105, comprising administering a UC28 targeted therapy and a differentiation agent to the subject if the UC28 expression in the sample is elevated as compared to the level of UC28 expression in a noncancerous epithelial reference.

114. (canceled)

115. The method of claim 107, wherein the UC28-specific antibody comprises an antibody that specifically binds to a polypeptide comprising the amino acid sequence set forth in SEQ ID NO:2, 3, or 4.

116. (canceled)

117. The method of claim 105, wherein the differentiation agent comprises sodium phenylbutyrate (SPB), onconase, troglitazone, or a hybrid polar cytodifferentiation agent.

118-121. (canceled)

122. The method of claim 105, wherein the UC28 targeted therapy comprises a UC28-specific antibody that binds to a polypeptide comprising an amino acid sequence as set forth in SEQ ID NOs: 2, 3 or 4.

123. The method of claim 105, further comprising the steps of (a) measuring the level of UC28 expression in a biological sample from a subject having a malignant prostate cancer and (b) determining whether the level of UC28 expression in a biological sample is elevated compared to the level of UC28 expression in a noncancerous epithelial reference.

124. The method of claim 122, further comprising measuring the level of prostate-specific antigen (PSA) expression, wherein a UC28 targeted therapy and/or a differentiation agent is an appropriate treatment for the subject if the level of PSA expression in the sample is elevated compared to the level of PSA expression in the noncancerous reference.

125. The method of claim 124, wherein the PSA expression comprises total PSA expression or free PSA expression

126. A method of detecting malignant prostate cancer in a sample obtained from a subject, the method comprising the steps of:
(a) measuring an amount of UC28 protein in the sample using a UC28-specific antibody that binds to a polypeptide comprising an amino acid sequence as set forth in SEQ ID NOs: 2, 3 or 4; and
(b) indicating that malignant prostate cancer is present in the sample if the level of UC28 protein in the sample is elevated as compared to the level of UC28 protein in a noncancerous epithelial reference sample.

127. The method of claim 126, wherein the malignant prostate cancer comprises bone metastatic prostate cancer.
